SEQUENCE LISTING (5 sequences)

SEQ ID NO: 1 - DNA, 18 bp, Artificial Sequence
  PCR primer
gtccaagctcagctaatt

SEQ ID NO: 2 - DNA, 18 bp, Artificial Sequence
  PCR primer
gcaggactctagaggatc

SEQ ID NO: 3 - DNA, 18 bp, Artificial Sequence
  PCR primer
aattagctgagcttggac

SEQ ID NO: 4 - DNA, 18 bp, Artificial Sequence
  PCR primer
gatcctctagagtcctgc

SEQ ID NO: 5 - protein, 4 aa, Artificial Sequence
  peptide
GRYL

(19) United States
(12) Patent Application Publication
(10) Pub. No.: US 2003/0162185 A1
(43) Pub. Date: Aug. 28, 2003

(54) Products comprising a support to which nucleic acids are fixed and their use as dna chips

(76) Inventors: Oleg Melnyk, Annoeulin (FR); Christophe Olivier, Lille (FR); Nathalie Ollivier, Lille (FR); David Hot, Lille (FR); Ludovic Huot, Villeneuve D'Ascq (FR); Yves Lemoine, Villeneuve D'Ascq (FR); Isabelle Wolowczuk, Lille (FR); Tam Huynh-Dinh, Paris (FR); Catherine Gouyette, Vanves (FR); Helene Gras-Masse, Merignies (FR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/149,249

(22) PCT Filed: Dec. 7, 2000
(86) PCT No.: PCT/FR00/03427

(30) Foreign Application Priority Data
Dec. 7, 1999 (FR) 99/15392

(51) Int. Cl. C12Q 1/68; C07H 21/04; C12M 1/34; B05D 3/00

(52) U.S. Cl. 435/6; 435/287.2; 536/24.3; 427/2.11

(57) ABSTRACT

The invention concerns products comprising a support whereon are fixed nucleic acids and their preparation method and use as DNA support. The invention also concerns functionalised supports, oligonucleotides and DNA's modified in position 5′ by a group selected in the group consisting of tartaric acid, serine, threonine, their derivatives and the α-oxoaldehyde group, and the methods for preparing them. The invention further concerns a method for fixing a nucleic acid on a support.

The present invention relates to products comprising a support to which nucleic acids are fixed, to the method for preparing them and to their use as a DNA chip. The present invention also relates to functionalized supports, to oligonucleotides and to DNAs modified in the 5′ position, and also to the methods for preparing them. The present invention also relates to a method for fixing the nucleic acid to a support.

Fixing a set of DNAs of known sequences to a support, in a very precise order, allows DNA chips to be obtained, which, by hybridization of the DNAs immobilized on the support with target oligonucleotides or nucleic acids, make it possible to determine the sequence of these target molecules or to monitor gene expression. There are many applications: discovering novel genes and novel medicinal products, performing diagnoses, studying toxicity, etc.

The method for obtaining DNA chips, which uses the fixing of DNA to a glass slide, mainly involves steps for preparing the glass slide (treatment of its surface with sodium hydroxide, then adsorption of polylysine or of polyethyleneimine onto the surface via ionic interactions; BURNS, N. L., Langmuir, 1995, 11, 2768-2776), depositing the DNA onto the glass slides thus prepared, and then heat treating and treating with UV irradiation so as to covalently attach the DNA to the glass surface.

The use of DNA normally makes it possible to obtain very specific hybridizations of high affinity compared to that which is obtained with relatively short oligonucleotides. Now, as indicated by D. J. LOCKHART in Nature Biotechnology, 1996, 14, 1675-1680, this is not the case. The method of producing the slides may explain this phenomenon. Specifically, the DNAs interact with the polylysine or the polyethyleneimine via ionic interactions involving negatively charged phosphodiester groups of the nucleic acids: this method of deposition thus limits the conformational freedom of the DNA and its accessibility. Furthermore, the heat treatment applied before UV irradiation degrades the DNA. In addition, the UV irradiation itself modifies pyrimidine bases (formation of hydrates or binding to the surface via the silanol functions of the glass, dimerization between several pyrimidines) although these bases are important for hybridization.

It thus appears that the method for attaching DNA to a glass slide described above is complex and subject to a lack of reproducibility. The advantages linked to the use of DNA instead of oligonucleotides are lost due to the method of treating the deposits. Finally, the DNA-glass surface attachment is not stable: the glass slides cannot be used more than 2 or 3 times in hybridization reactions.

Another method for immobilizing DNA or oligonucleotides on glass slides has been described by M. BEIR (Nucleic Acids Research, 1999, 27, 1970-1977). It consists of forming an amide bond between the DNA or oligonucleotide and the support. To this effect, glass slides silanized with aminopropyltrimethoxysilane are derivatized with a bifunctional coupling agent which provides an activated carboxylic acid function. Coupling agents which can be used are, for example, phenylenediisothiocyanate, disuccinimidyl carbonate, disuccinimidyl oxalate or dimethylsuberimidate described by G. T. HERMANSON in Bioconjugate Techniques, 1996, Academic Press (San Diego, Calif.). The DNAs or oligonucleotides, modified in the 5′ position by an amine function, are deposited, under basic conditions, onto the functionalized glass slides. The slides obtained appear to be quite stable to recycling.

However, this method comprises several drawbacks: first of all, the use of a bifunctional agent in the support may lead to crosslinking of this agent onto the surface, and therefore to a partial loss of charge of the surface. The use of isothiocyanate or N-hydroxysuccinimide esters produces a risk of hydrolysis of these functions during the storage of the slides or during the fixing, under basic conditions, of the DNA or of the oligonucleotide to the support, i.e., once again, to a loss of charge of the surface. Finally, the authors specify that it is necessary to block the reactive functions of the support after the DNA or the oligonucleotides have been fixed: clearly, not all the reactive functions have reacted. This may be explained by the slowness of the coupling kinetics, which lead to a low rate of attachment and to partial hydrolysis of the reactive functions.

Other techniques consist in using a polyacrylamide gel as support and in immobilizing oligonucleotides thereon via a hydrazone bond (D. GUSCHIN, et al., Anal. Biochem., 1997, 250, 203-211). Such an immobilization involves the synthesis of an oligonucleotide functionalized in 3′ with a 3-methyluridine, periodate oxidation thereof to transform the diol of the uridine into dialdehyde, activation of the polyacrylamide gel with hydrazine in order to create hydrazide functions, and then deposition of the oxidized oligonucleotide onto the activated gel.

This method, which does not apply to DNAs, is complex and involves periodate oxidation of a ribose in the 3′ position; the products thus formed are known to be unstable (R. L. P. ADAMS, et al., The Biochemistry of the nucleic acids, tenth edition, 1986, Chapman and Hall, New York). D. GUSCHIN, et al. (ibid) specify, moreover, that oxidized oligonucleotides can only be stored for a week at 4° C.

With regard to the immobilization of DNA, these same authors (D. GUSCHIN, et al., ibid) describe steps consisting in depurinating the DNA in formic acid, in precipitating in acetone and then in depositing onto a hydrazine-activated polyacrylamide gel. In this method, the initial structure of the DNA is therefore denatured to allow its immobilization, which affects its subsequent hybridization capacity. In addition, depurinating the DNA in acid medium leads to polypentose-phosphate diester regions linking pyrimidine oligonucleotide regions: now, phosphate diester bonds are fragile, readily giving β-elimination in the presence of bases. The DNA thus immobilized is therefore relatively unstable.

Besides polyacrylamide gels, it has also been proposed to immobilize the DNA on agarose gels. To this effect, P. N. GILLES (Nature Biotechnology, 1999, 17, 365-370) prepares a glycosal agarose gel with streptavidin, reduces it with sodium cyanoborohydride, synthesizes DNAs carrying a biotin in the 5′ position, and then deposits them on the gel. A major drawback of this method is the noncovalent nature of the DNA-support binding.

In another technical field, U.S. Pat. No. 4,874,813 describes the attachment of glycoproteins to a solid support via a hydrazone bond, the support being functionalized with a hydrazide in the glycoprotein carrying an aldehyde function, introduced by oxidation of the carbohydrate component of the glycoprotein, which carries a 1,2-diol function. This technique cannot, however, be extrapolated to DNAs, which naturally lack 1,2-diol functions, nor to nucleic acids, whatever they are. Specifically, while the glycoproteins used in U.S. Pat. No. 4,874,813 are obtained with amounts of the order of tens of milligrams, nucleic acids are, themselves, handled on the microgram scale. It is therefore necessary to compensate for this dilution factor by using functional groups which are more reactive than aldehydes obtained by oxidation of sugars. Finally, a nucleic acid carrying an aldehyde function at its end is relatively unstable, is in particular subject to oxidation reactions in the presence of air, and readily gives rise to the formation of imines when it is placed together with enzymes, for example during amplification reactions.

Thus, the inventors have given themselves the aim of overcoming the drawbacks of the prior art and of providing a method for fixing nucleic acids, in particular DNA or oligonucleotides, to a support, which in particular satisfies the following criteria:

the method is simple at the experimental level, reproducible and inexpensive;

it allows nucleic acid to be attached to a support via covalent bonds;

it allows nucleic acid-support attachment which is very stable under hybridization and washing conditions, limiting desorption of the nucleic acid and allowing the production, when the nucleic acid is DNA, of DNA chips which are reusable in many hybridization cycles;

it uses a modified nucleic acid which is stable and the production of which is simple;

it uses a support derivatized with a stable, nonhydrolyzable function;

it involves, in the attachment between the nucleic acid and the support, very reactive functions, compensating for the low concentration of partners;

it does not involve any denaturation of the structure of the nucleic acid, the latter remaining optimal for subsequent hybridization reactions.

The subject of the present invention is a product of formula (I):

SP[Aᵢ(Yᵢ—Z—CO—M)ₙ]ₘ (I)

in which:

Z represents a group of formula

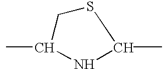

or a group —X—N═CH—, X representing a group —CH₂—O—, —CH₂—NH— or —NH—,

is equal to 0 or to 1,

n is between 1 and 16, n being equal to 1 when i is equal to 0,

m is greater than or equal to 1,

SP represents a support,

A represents a spacer arm,

Y represents a function which provides the attachment between A and Z, and

M represents a nucleic acid attached to the adjacent group —CO— via its 3′ or 5′ end.

For the purposes of the present invention, the term “nucleic acid” is intended to mean a DNA, an RNA or an oligonucleotide, the latter corresponding to a series of approximately 1 to 50 bases, said nucleic acid comprising natural nucleosides (A, C, G, T or U) or nucleosides modified at the level of the base (heterocycle), of the sugar and/or of the phosphodiester bond. Said nucleic acid may therefore also consist of a PNA (Peptide Nucleic Acid).

In formula (I) above, when Z represents a group —X—N═CH—, the latter represents an oxime bond when X is a group —CH₂—O— and a hydrazone bond when X is a group —CH₂—NH— or —NH—. The product of formula (I) may also comprise a thiazolidine bond when Z represents a group of formula:

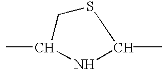

Depending on the nature of the group Z, the product of formula I according to the present invention may therefore correspond to the products of formulae (Ia) and (Ib) represented below, in which SP, A, Y, X, M, i, n and m are as described above:

SP[Aᵢ(Yᵢ—X—N═CH—CO—M)ₙ]ₘ (Ia)

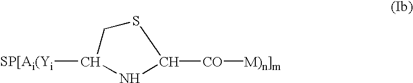

The product of formula (I) according to the present invention is such that the bond between the nucleic acid M and the support SP is very stable.

In formula (I), n is advantageously equal to 1 and A preferably represents a linear or branched carbon-based chain comprising from 2 to 100 carbon atoms, preferably from 5 to 50 carbon atoms, and optionally comprising from 1 to 35 oxygen or nitrogen atoms and from 1 to 5 silicon, sulfur or phosphorus atoms.

SP advantageously represents a solid support, preferably made of glass, of silicon or of a synthetic polymer, such as nylon, polypropylene or polycarbonate. SP may also advantageously represent a nonsolid support, such as a natural polymer (for example a polysaccharide such as cellulose or mannan, or a polypeptide), a synthetic polymer (for example a copolymer of N-vinylpyrrolidone and of acrylic acid derivatives), a liposome or a lipid. SP may advantageously represent a transfection vector, i.e. an organic compound (lipid or peptide for example) which is permeable at the cell membrane level.

According to an advantageous embodiment of the product of formula (I) according to the present invention, SP is a solid support, i is equal to 1, n is equal to 1 and M is a DNA, said product constituting a DNA chip.

In such a DNA chip, the m molecules M present in the chip may be identical, but are preferably different from one another. The DNA chips according to the present invention are reusable in many hybridization cycles, the attachment between the DNA and the support being very stable under the hybridization and washing conditions, which considerably limits the possibilities of desorption of the DNA.

According to another advantageous embodiment of the product of formula (I) according to the present invention, SP represents a glass support, i is equal to 1, n is equal to 1, A represents a spacer arm of formula —Si—(CH₂)₃— and Y represents an amide function —NH—CO—.

The subject of the present invention is also the use of the product of formula (I) above, when SP is a solid support, as a nucleic acid chip, such as a DNA or oligonucleotide chip.

A subject of the present invention is also a method for preparing a product of formula (I) above, which comprises reacting n×m molecules of formula M—CO—CHO with a product of formula SP[Aᵢ(Yᵢ—B—NH₂)ₙ]ₘ, SP, A, Y, i, n, m and M being as defined above and B representing a group —CH₂—O—, —CH₂—NH—, —NH— or —CH(CH₂SH)—.

The molecules of formula M—CO—CHO correspond to nucleic acids carrying an α-oxoaldehyde (—CO—CHO) function at their 5′ or 3′ ends.

A subject of the present invention is also a method for fixing, via covalent attachment, at least one nucleic acid M to a support SP, so as to produce a product of formula (I) as described above, characterized in that it comprises the following steps:

i) introducing an α-oxoaldehyde function at one end (5′ or 3′ end) of said nucleic acid, and

ii) reacting the functionalized nucleic acid obtained in step i) with a support modified by a function selected from the group consisting of hydrazine, hydrazine-derived, hydroxylamine and β-aminothiol functions.

As a hydrazine-derived function, mention may be made, for example, of hydrazide functions, i.e. a hydrazine substituted with at least one acyl or carbonyl group.

Step ii) above results in the formation of a hydrazone bond (when the support carries a hydrazine function or hydrazine-derived function), an oxime bond (when the support carries a hydroxylamine function) or a thiazolidine bond (when the support carries a β-aminothiol function) between the nucleic acid and the support.

Particularly advantageously, the method according to the present invention uses a support modified by a function which is stable within a wide pH range. The functions which are involved during the attachment, namely the α-oxoaldehyde function carried by the nucleic acid and the function carried by the support, are very reactive. In addition, the method according to the present invention does not involve any denaturation of the structure of the nucleic acid, which remains optimal for subsequent hybridization reactions, when the product of formula (I) obtained using the method according to the present invention is used as a DNA chip.

According to an advantageous embodiment of the method according to the present invention, an α-oxoaldehyde function is introduced at one of the ends of said nucleic acid via the following steps:

a) introduction of a group selected from the group consisting of tartaric acid, serine and threonine, and derivatives thereof, at one of the ends of an oligonucleotide,

b) hybridization of the oligonucleotide obtained in step a) with said nucleic acid,

c) elongation of said oligonucleotide,

d) reiteration of steps b) and c) at least once,

e) periodate oxidation of the nucleic acid obtained in step d), modified at one of its ends by a group selected from the group consisting of tartaric acid, serine and threonine, and derivatives thereof, and

f) isolation of a nucleic acid modified at one of its ends by an α-oxoaldehyde function.

According to another advantageous embodiment of the method according to the present invention, an α-oxoaldehyde function is introduced at one end of said nucleic acid via the following steps:

a) introduction of a group selected from the group consisting of tartaric acid, serine and threonine, and derivatives thereof, at one of the ends of an oligonucleotide,

b) periodate oxidation of the oligonucleotide obtained in step a),

c) hybridization of the oligonucleotide obtained in step b), carrying an α-oxoaldehyde function at one of its ends, with said nucleic acid,

d) elongation of said oligonucleotide,

e) reiteration of steps c) and d) at least once, and

f) isolation of a nucleic acid modified at one of its ends by an α-oxoaldehyde function.

In the methods above, it is clearly understood that the step of hybridization of the oligonucleotide with the nucleic acid is carried out after a step of denaturation of said nucleic acid, as known by those skilled in the art.

The steps of hybridization between an oligonucleotide and a nucleic acid, of elongation of said oligonucleotide and reiteration of these steps constitute cycles of amplification of the nucleic acid, the oligonucleotide being used as a primer for these amplifications, which may be carried out using the “PCR” (Polymerase Chain Reaction) technique well known to those skilled in the art, described, for example, in Molecular Cloning, second edition, J. SAMBROOK, E. F. FRITSCH and T. MANIATIS (Cold Spring Harbor Laboratory Press, 1989).

The step of elongation of the oligonucleotide, after it has been hybridized with the nucleic acid, is carried out in a suitable buffer medium, in the presence of the nucleotide bases required for forming nucleic acid.

As examples of tartaric acid derivatives which can be used in the methods described above, mention may be made of diacetyltartaric acid, di-para-toluyltartaric acid, metatartaric acid, dimethyl tartrate, disuccinimidyl tartrate, tartaric anhydride and diacetyltartaric anhydride.

Advantageously, a group selected from the group consisting of tartaric acid, serine and threonine, and derivatives thereof, is introduced at one of the ends of an oligonucleotide (step a) of the methods described above) via an amide bond. The group selected from the group consisting of tartaric acid, serine and threonine, and derivatives thereof, is preferably attached to the oligonucleotide via a spacer arm attached, via one of its ends, to said oligonucleotide and carrying, at its other end, an amine function.

In the method for fixing a nucleic acid M to a support SP described above, the nucleic acid is preferably a DNA. In this case, the oligonucleotide which hybridizes with this nucleic acid is an oligodeoxynucleotide primer which may be specific or universal. The DNA may be obtained by amplification of genomic DNA or by amplification of DNA inserted into a vector, for example the M13 phage. In the case of DNA obtained by amplification of genomic DNA, a first amplification with a series of specific primers may be followed by amplification using universal primers (see, for example, J. R. POLLACK, in Nature Genetics, 1999, 23, 41-46). In this case, two primers make it possible to amplify a considerable number of different DNAs. It is particularly advantageous to modify these universal primers with a group chosen from tartaric acid, serine and threonine, and derivatives thereof, or the product of oxidation of these groups, namely an α-oxoaldehyde function. In the case of DNA obtained by amplification of DNA inserted into a vector, here again, a considerable number of different DNAs may be amplified through using universal primers functionalized with a group chosen from tartaric acid, serine and threonine, and derivatives thereof, or the product of oxidation of these groups.

A subject of the present invention is also an oligonucleotide modified in the 5′ position by a group selected from the group consisting of tartaric acid, serine and threonine, and derivatives thereof, and the α-oxoaldehyde group.

Such an oligonucleotide may advantageously be used as a primer in nucleic acid elongation or amplification reactions, in order to obtain nucleic acids modified in the 5′ position by the groups carried by said oligonucleotide.

A subject of the present invention is also a method for preparing such an oligonucleotide, characterized in that it comprises a step for introducing a group selected from the group consisting of tartaric acid, serine and threonine, and derivatives thereof, at the 5′ position of said oligonucleotide, this step being followed, when said oligonucleotide is modified by an α-oxoaldehyde group, by periodate oxidation of said oligonucleotide.

A subject of the present invention is also a DNA modified in the 5′ position by a group selected from the group consisting of tartaric acid, serine and threonine, and derivatives thereof, and the α-oxoaldehyde group.

Such a DNA may advantageously be used in the method according to the present invention for fixing, via covalent bonding, at least one nucleic acid M to a support SP, as described above.

In fact, the tartaric acid, serine and threonine groups can be readily converted to the α-oxoaldehyde function via a periodate oxidation reaction. It is particularly advantageous to use a DNA modified by an α-oxoaldehyde function since this function is very stable and very reactive, and in any event, much more stable and reactive than an aldehyde function. These considerations also apply to the oligonucleotides according to the present invention, modified by an a-oxoaldehyde function. Thus, the nucleic acids, whatever they are (in particular DNAs or oligonucleotides), can be conserved without oxidizing or degrading, in particular in the presence of air (independently of the position of their functionalization at the 3′ or 5′ end). They give rise to very stable bonds with supports carrying corresponding reactive functions, as described above. In addition, and unlike nucleic acids functionalized with an aldehyde, they do not induce imine formation with the enzymes present during nucleic acid elongation or amplification reactions and, in the case of a PCR amplification using Taq polymerase, they do not interact with the dithiothreitol, an enzyme-preserving agent.

A subject of the present invention is also a method for preparing a DNA modified at the 5′ position by a group selected from the group consisting of tartaric acid, serine and threonine, and derivatives thereof, and the α-oxoaldehyde group, as described above, characterized in that it comprises the following steps:

a) introduction of a group selected from the group consisting of tartaric acid, serine and threonine, and derivatives thereof, at the 5′ position of an oligonucleotide,

b) hybridization of the oligonucleotide obtained in step a) with a DNA,

c) elongation of said oligonucleotide,

d) reiteration of steps b) and c) at least once,

or, when said DNA is modified by an α-oxoaldehyde group, steps a) to f) of the methods described above in connection with the method according to the present invention relating to the introduction of an α-oxoaldehyde function at one of the ends of a nucleic acid.

The step of elongation of the oligonucleotide, after it has been hybridized with the DNA, is carried out in a suitable buffer medium, in the presence of the deoxynucleotide bases required for forming DNA.

A subject of the present invention is also a functionalized support of formula (II):

SP[Aᵢ(Yᵢ—B—NH₂)ₙ]ₘ (II)

in which SP, A, Y, B, i, n and m are as defined above.

Such a support carries a hydrazine function (when B represents a group —CH₂—NH— or —NH—), a hydroxylamine function (when B represents a group —CH₂—O—) or a β-aminothiol function (when B represents a group —CH(CH₂SH)—).

The functionalized support of formula (II) may advantageously be used in the method according to the present invention for fixing, by covalent attachment, at least one nucleic acid M to a support SP, as described above.

A subject of the present invention is also a method for preparing the functionalized support of formula (II), in which i is equal to 1, n is equal to 1 and SP represents a glass support, characterized in that it comprises the following steps:

silanizing the glass support,

grafting, onto said silanized glass support, a function selected from the group consisting of the hydrazine, hydrazine-derived, hydroxylamine and β-aminothiol functions.

The step of silanizing the support is preferably carried out using aminopropyltrimethoxysilane.

Particularly advantageously, said grafting of a hydrazine function is carried out using hydrazinoacetic acid, said grafting of a hydrazine-derived function is carried out using triphosgene and hydrazine, said grafting of a hydroxylamine function is carried out using aminooxyacetic acid and said grafting of a β-aminothiol function is carried out using α-amino-β-mercaptopropionic acid.

A subject of the invention is also a method for controlling the quality of the support of formula (II) as defined above, characterized in that it comprises the following steps:

bringing the support into contact with a fluorescent probe, for example rhodamine, derivatized with an α-oxoaldehyde function,

washing the support obtained at the end of the previous step, and

analyzing the fluorescence from this support.

This method makes it possible to analyze the homogeneity of the functionalization of the support, i.e. the spatial distribution of the terminal —B—NH₂ groups (cf. formula (II) above).

A subject of the invention is also a method for quantifying the functionality of the support of formula (II) as defined above, characterized in that it comprises the following steps:

bringing the support into contact with a fluorescent probe, for example rhodamine, derivatized with an α-oxoaldehyde function,

washing the support obtained at the end of the previous step,

hydrolyzing the attachment between the support and the fluorescent probe, in strong acid medium or by enzymatic hydrolysis (nuclease, protease, etc), and

measuring the amount of fluorescence released into solution at the end of this hydrolysis.

This method makes it possible to quantify the number of functional sites accessible to the fluorescent probe, on the support of formula (II) above.

A subject of the present invention is also a kit for preparing a DNA chip as described above, characterized in that it comprises the following elements:

at least one functionalized support of formula (II) according to the present invention,

a plurality of oligodeoxynucleotide primers which are modified either in the 3′ position, or in the 5′ position, or in the 3′ position for a part of said primers and in the 5′ position for the other part of said primers, by tartaric acid, serine and threonine, and derivatives thereof, and the α-oxoaldehyde group,

reagents and buffers suitable for carrying out reactions of elongation and/or of amplification of said DNA, and

when said oligodeoxynucleotide primers are modified by a group selected from the group consisting of tartaric acid, serine and threonine, and derivatives thereof, reagents suitable for carrying out a periodate oxidation reaction.

A subject of the invention is also the use of the DNA chip as defined above in the following fields of application:

in combinatorial chemistry, in particular for high throughput screening of molecules. This may involve, for example, screening bacterial strains, identifying contaminants or identifying genes;

as a diagnostic tool; and

for sorting molecules.

A subject of the invention is also a method for sorting molecules, which uses the DNA chip as defined above, and also the sorted molecules which can be obtained using this method.

Besides the above arrangements, the invention also comprises other arrangements which will emerge from the following description, which refers to examples of implementation of the methods which are the subjects of the present invention, and also to the attached figures, in which:

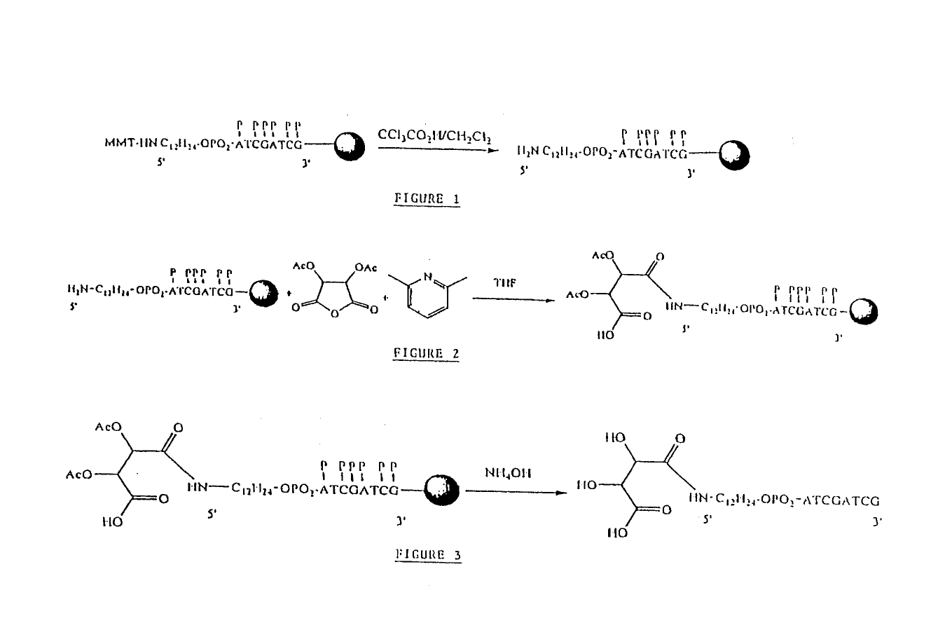

FIGS. 1 and 10 represent the deprotection of the MMT group carried, respectively, by the oligonucleotide prepared in accordance with example 1 and by the oligonucleotide prepared in accordance with example 2,

FIGS. 2, 5 and 7 represent, respectively, the coupling of an oligonucleotide with (+)-diacetyl-L-tartaric anhydride, disuccinimidyl tartrate and trifluoroacetyl-serine, in accordance with example 1,

FIGS. 3, 6 and 8 represent reactions of aminolysis of an oligonucleotide immobilized on a solid support, in accordance with example 1,

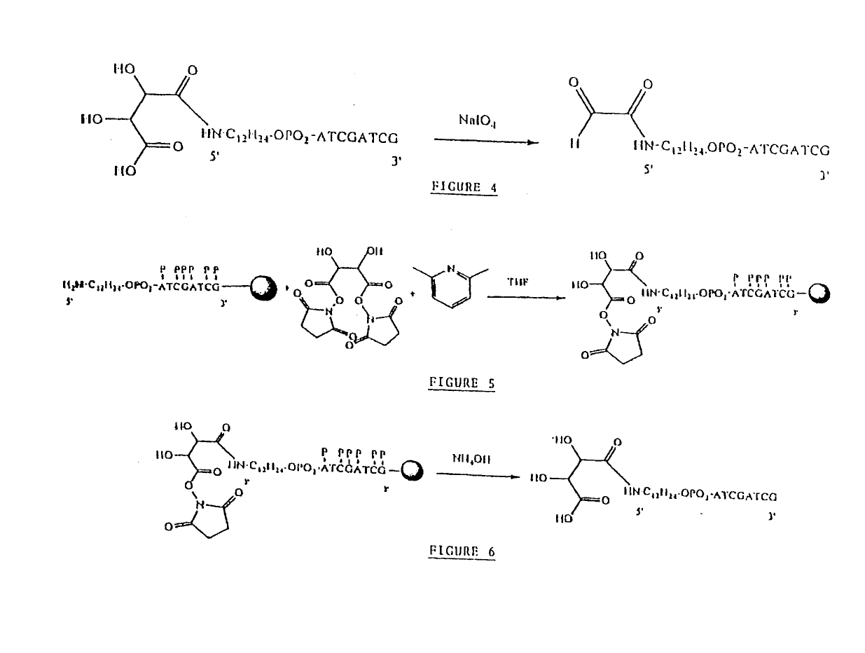

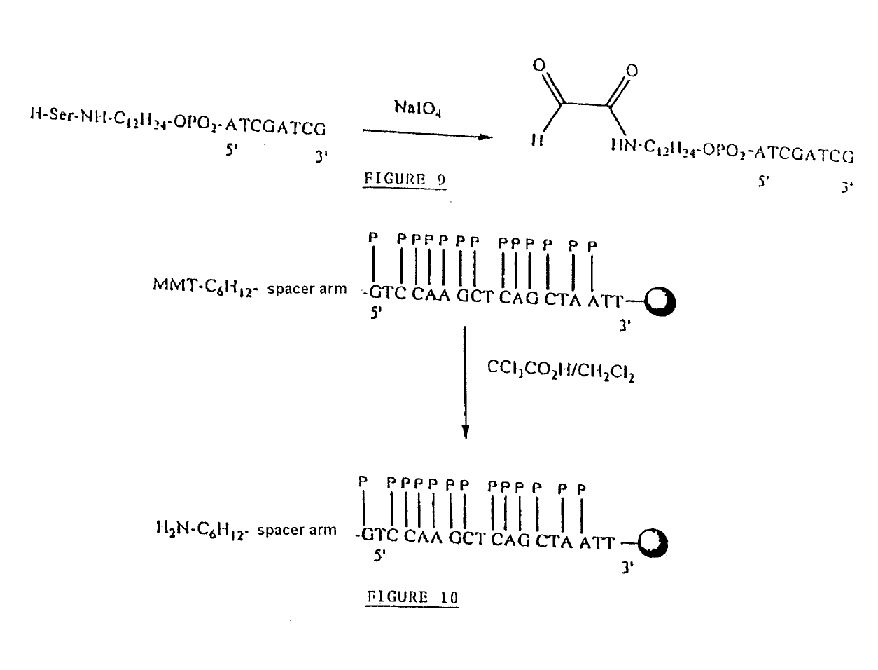

FIGS. 4 and 9 represent periodate oxidation reactions, in accordance with example 1,

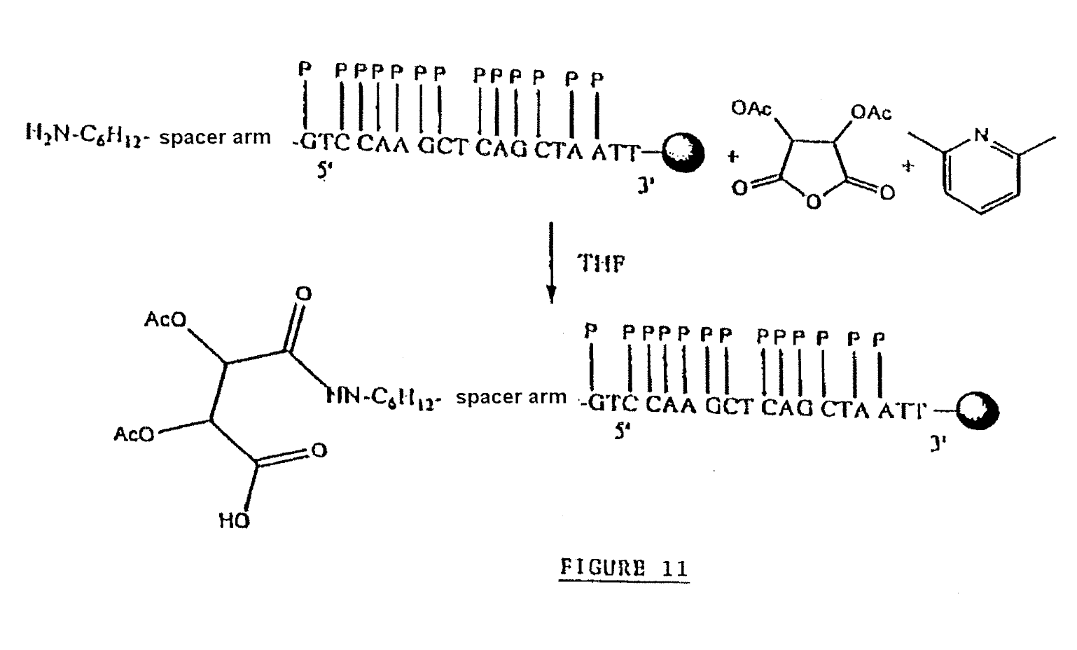

FIG. 11 represents the coupling of primer 1 with (+)-diacetyl-L-tartaric anhydride,

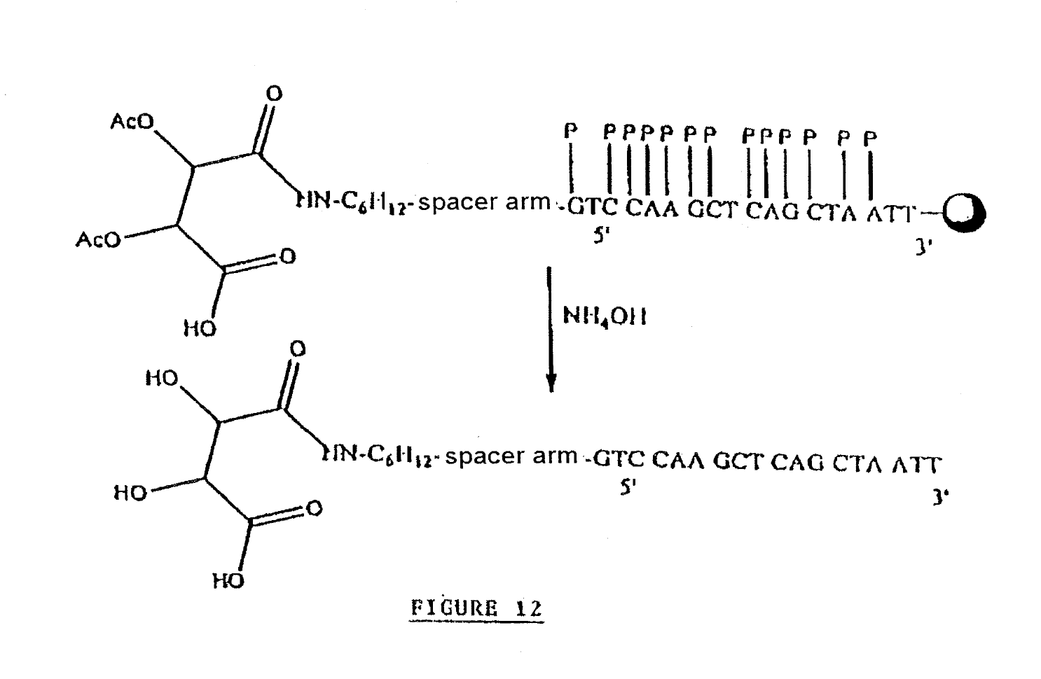

FIG. 12 represents the reaction of aminolysis of primer 1 immobilized on a solid support, in accordance with example 2,

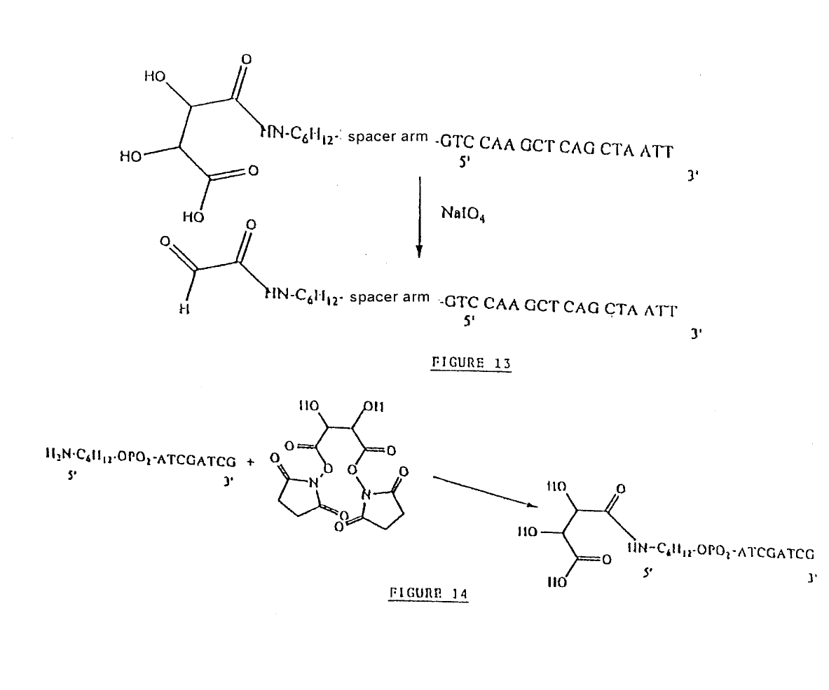

FIG. 13 represents the reaction of periodate oxidation of primer 1, in accordance with example 2,

FIG. 14 represents the coupling of an oligonucleotide with disuccinimidyl tartrate, in accordance with example 3,

FIG. 15 represents the synthesis of a glass surface functionalized with a hydrazide function, in accordance with example 5,

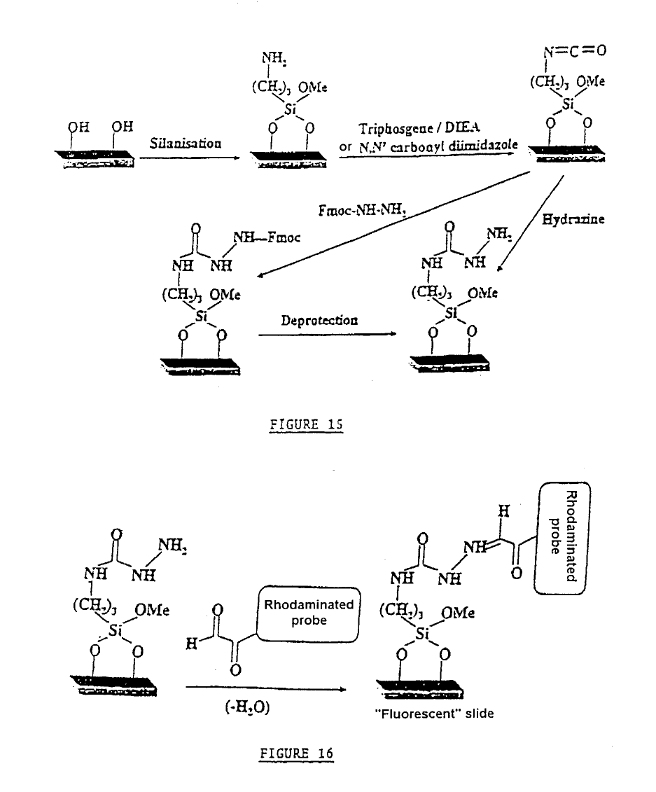

FIG. 16 represents the ligation of a fluorescent probe (rhodaminated probe) to a glass surface functionalized with a hydrazide group, for controlling the quality of this surface, in accordance with example 5,

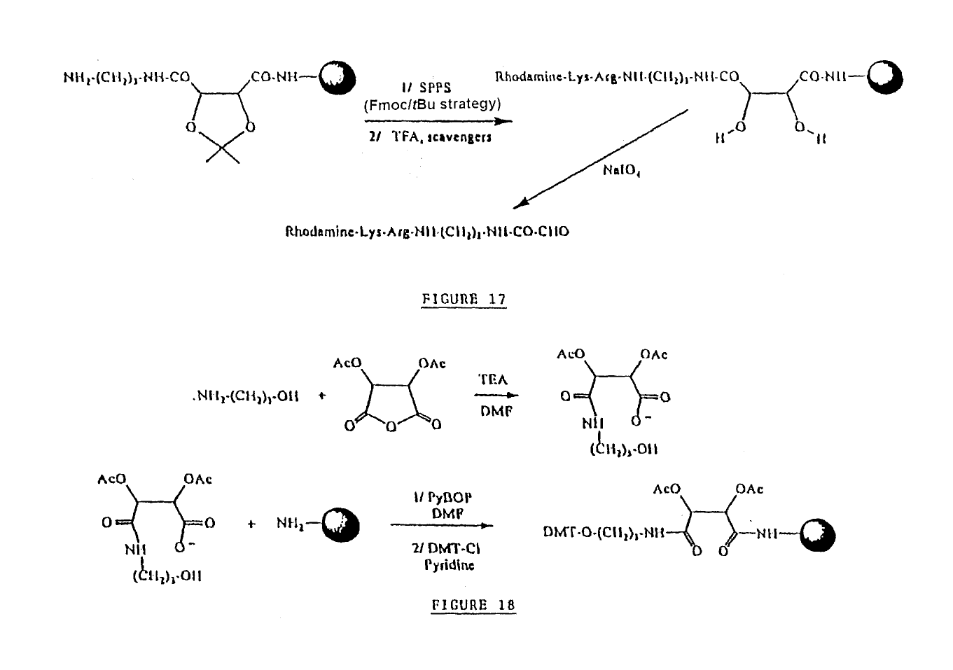

FIG. 17 represents the synthesis of a rhodaminated peptide functionalized with an α-oxoaldehyde group, in accordance with example 5, and

FIG. 18 represents the preparation of a functionalized solid support suitable for synthesizing oligonucleotides carrying, at their 3′ end, an α-oxoaldehyde function, in accordance with example 4.

In FIGS. 1, 2, 3, 5, 6, 7, 8, 10, 11 and 12, when the oligonucleotide immobilized on the support is protected, the β-cyanoethyl protection of the phosphodiester linkage is omitted in the interest of greater clarity.

It should be clearly understood, however, that these examples are given purely by way of illustration of the subject of the invention, of which they in no way constitute a limitation.

### EXAMPLE 1

### Production, by Solid-phase Chemistry, of Oligonucleotides Modified in the 5′ Position by an α-oxoaldehyde Function

In this example, the oligonucleotides all have the following sequence: ATCGATCG.

1) Synthesis of the Oligonucleotide

An oligonucleotide of sequence ATCGATCG is synthesized in solid phase, for example on a CPG (controlled pore glass) support, according to the technique described in “Oligonucleotide Synthesis: a practical approach”, ed. M. J. GAIT, IRL Press, Oxford, 1984, or in “Protocols for oligonucleotides and analogs: synthesis and properties”, ed. S. AGRAWAL, Humana Press, Totowa N.J., 1993, or by A. ELLINGTON and J. D. POLLARD in “Current Protocols in Molecular Biology”, 1998, 2.11.1-2.11.25, John Wiley & Sons Inc., New York. The synthesis follows a conventional strategy (5′ hydroxyls protected with dimethoxytrityl groups, cyanoethoxyphosphoramidite chemistry). The bases are protected with acyl groups, which will be labile at the end of the synthesis during the aminolysis.

Once the oligonucleotide has been synthesized, the 5′ hydroxyl is deprotected and is coupled with an aminated spacer arm of formula C₁₂H₂₄—OPO₂—, protected with a monomethoxytrityl (MMT) group. The MMT group will be removed at the last minute, just before coupling with a tartaric acid derivative, according to the reaction scheme represented in FIG. 1, in which the group P represents the nucleotide base-protecting group (benzoyl for bases A and C, isobutyryl for base G). For this purpose, 0.8 μmol of oligonucleotide on a support, on the oligonucleotide synthesizer, are mixed with 3% trichloroacetic acid in dichloromethane, for 5 minutes 30 seconds, performing two intermediate washes with CH₃CN (acetonitrile) in order to remove the yellow coloration. The “supported” oligonucleotide (i.e. the oligonucleotide immobilized on the support) is then washed with CH₃CN and dried under argon, and then with compressed air.

2) Introduction of the α-oxoaldehyde Function Using (+)-diacetyl-L-tartaric Anhydride

a) Coupling of (+)-diacetyl-L-tartaric anhydride (FIG. 2)

This reaction is represented in FIG. 2, in which Ac represents acetyl groups. 0.4 μmol of oligonucleotide on a support are transferred into an empty oligonucleotide synthesis column comprising, at both its ends, two gas-tight syringes. 4 μl (85.86 eq) of 2,6-lutidine dissolved in 80 μl of THF (tetrahydro-furan) are introduced into the column via one of the two syringes. The oligonucleotide is left in contact with this solution while 4.012 mg (46.4 eq) of (+)-diacetyl-L-tartaric anhydride (hereinafter designated “tartaric anhydride”) are dissolved in 80 μl of THF. The latter solution is introduced, in turn, into the column, which is agitated manually for 5 minutes. The supported oligonucleotide is then washed several times (6 cycles) with THF on the synthesizer. It is dried with argon and with compressed air before undergoing a second coupling for 10 minutes under the same conditions. Finally, it is washed again with THF in the synthesizer and dried with argon and then with compressed air.

b) Aminolysis (FIG. 3)

2 plastic syringes are placed at the 2 ends of the column containing the oligonucleotide on a support. 1 ml of NH₄OH (aqueous ammonia) at 32% in water is placed in the first. A mechanical system allows the plunger of this syringe to be pushed in order to deliver 250 μl of fresh aqueous ammonia every 15 minutes. The oligonucleotide in solution is thus recovered in the other syringe at the other end. This solution is then transferred into a screw-cap eppendorf and placed in an incubator at 55° C. overnight. The solution is then cooled and 10 μl are removed in order to assay the oligonucleotide obtained (22.9 OD in 1,000 μl). The ammoniacal solution is evaporated off and the residue is taken up in 400 μl of water.

c) Analyses and purification by RP-HPLC

For the analytical RP-HPLC, 10 μl of the stock solution are set aside (0.57 OD, i.e. 18.90 μg of oligonucleotide), lyophilized and taken up in 400 μl of water. 83.5 μl of the latter solution are injected onto a 250×4.6 mm C18 hypersil (30° C., detection at 260 nm, buffer A: 99/1 by volume 100 mM TEAA in water, pH=6.5/CH₃CN, buffer B: 95/5 by volume CH₃CN/H₂O, gradient 1 to 40% of B in 28 minutes, flow rate 1 ml/min). A distinctly main peak with a purity of 74.75% is observed.

For the preparative RP-HPLC, the remainder of the stock solution is purified on an SP 250/10 Nucleosil 300/5 C18 column (ambient temperature, detection at 254 nm, buffer A: 10 mM TEAA in water, buffer B: CH₃CN, gradient 5 to 40% of B in 20 minutes, flow rate 5.5 ml/min). The fractions corresponding to the main product are isolated. The fractions are pooled and evaporated in a rotary evaporator, taken up in H₂O, frozen and lyophilized.

d) Quantification, analyses by RP-HPLC and by electrospray mass spectrometry

The residue is taken up in 1,000 μl of water. Assaying at 260 nm makes it possible to calculate an amount of nucleotide of 256.28 μg. Analysis by RP-HPLC: 51 μl of the stock solution are injected onto a 250×4.6 mm C18 hypersil under the same analytical conditions as above. A product with a purity of 99.14% is obtained. Electrospray mass spectrometry analysis: 5 μl of the oligonucleotide in solution in H₂O/20% i-PrOH/1% TEA are injected at a concentration of 10 pmol/μl. Electrospray mass spectrometry analysis: buffer comprising 20% i-PrOH in H₂O is continuously infused. 5 μl of the oligonucleotide in solution in H₂O/20% i-PrOH/1% TEA are injected at a concentration of 10 pmol/μl. The analysis is carried out in negative ionization mode and the voltage cone is 50V. The flow rate is 10 μl/min and the temperature is 70° C. An oligonucleotide corresponding to the following formula is obtained, the observed mass of which is 2803.0:

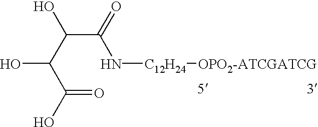

e) Periodate oxidation (FIG. 4)

The reaction was carried out on 181.5 μg of modified oligonucleotide. 1.29 mg of NaIO₄ (sodium periodate, M=213.89) are dissolved in 1206.73 μl of 100 mM sodium acetate buffer, pH=4.04. 100 μl of the latter solution are removed (i.e. 0.1069 mg of NaIO₄) and are deposited onto the oligonucleotide residue. The final concentration of oligonucleotide is 0.65 mM, that of NaIO₄ is 5 mM, i.e. 7.7 eq of NaIO₄ relative to the oligonucleotide. The medium is stirred at ambient temperature for 1 h 30 min (RP-HPLC monitoring: 4 μl of reaction mixture are removed and diluted with 96 μl of water; 250×4.6 mm C18 hypersil column; 30° C.; detection buffer A: 99/1 by volume 100 mM TEAA, pH=6.5/CH₃CN, buffer B: 95/5 CH₃CN/H₂O, gradient 1 to 40% of B in 28 minutes, 1 ml/min). 1 hour 30 min later, the excess NaIO₄ is consumed with 2 eq of tartaric acid relative to NaIO₄, i.e. 10 μl of a solution containing 0.9 mg of tartaric acid dissolved in 60 μl of water. The reaction medium is frozen at −30° C., before being purified by RP-HPLC.

f) Purification of the oxidized product by RP-HPLC

The reaction medium is taken up in 2 ml of water and the tube which contained the reaction medium is rinsed again with 2 ml of water, and this is injected onto a C18 hyperprep column (30° C., detection 260 nm, buffer A: 99/1 by volume 100 mM TEAA, pH=6.5/CH₃CN, buffer B: 95/5 by volume CH₃CN/H₂O, gradient 0 to 40% of B in 86 minutes, 3 ml/min, detection at 260 nm). The product is frozen and lyophilized. The residue is taken up in 3 ml of water. Assaying at 260 nm gives 112.6 μg of oligonucleotide, i.e. a periodate oxidation yield of 63.77%. Analysis by electrospray mass spectrometry: buffer comprising 20% i-PrOH in H₂O is infused continuously. 5 μl of the oligonucleotide in solution in H₂O/20% i-PrOH/1% TEA are injected at a concentration of 10 pmol/μl. The analysis is carried out in negative ionization mode and the voltage cone is 50V. The flow rate is 10 μl/min and the temperature is 70° C. Results: [M−3H]³⁻ 908.4, [M−4H]⁴⁻ 681.1, [M−5H]⁵⁻ 544.7. An oligonucleotide corresponding to the following formula is obtained:

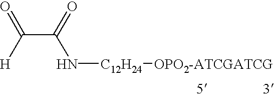

3) Introduction of the α-oxoaldehyde Function Using Disuccinimidyl Tartrate

a) Coupling of the disuccinimidyl tartrate (FIG. 5)

0.4 μmol of oligonucleotide on a support are transferred into an empty oligonucleotide synthesis column comprising, at both its ends, two gas-tight syringes. 4 μl (85.86 eq) of 2,6-lutidine dissolved in 80 μl of THF are introduced into the column via one of the two syringes. The oligonucleotide is left in contact with this solution while 6.38 mg (46.4 eq) of disuccinimidyl tartrate are dissolved in 80 μl of THF. The latter solution is introduced, in turn, into the column, which is agitated manually for 5 minutes. The supported oligonucleotide is then washed several times (6 cycles) with THF on the synthesizer. It is dried with argon and with compressed air before undergoing a second coupling for 10 minutes under the same conditions. Finally, it is washed again with THF in the synthesizer and dried with argon and then compressed air.

b) Aminolysis (FIG. 6)

2 plastic syringes are placed at the two ends of the column containing the oligonucleotide on a support. 1 ml of NH₄OH at 32% in water is placed in the first. A mechanical system allows the plunger of this syringe to be pushed in order to deliver 250 μl of fresh aqueous ammonia every 15 minutes. The oligonucleotide in solution is thus recovered in the other syringe, at the other end. This solution is then transferred into a screw-cap eppendorf and placed in an incubator at 55° C. overnight. The solution is then cooled and 10 μl are removed in order to assay the oligonucleotide obtained (26.9 OD in 1,000 μl). The ammoniacal solution is evaporated off and the residue is taken-up in 400 μl of water.

c) Analysis and purification by RP-HPLC

The crude is analyzed by analytical RP-HPLC under the same conditions as above. 1 main peak with a purity of 30.1% is obtained.

d) Analysis by electrospray mass spectrometry

Using conventional analytical conditions, the product is 97% pure. Observed M: 2803.5 (ES-MS).

e) Periodate oxidation

The procedure is carried out as indicated above.

4) Introduction of the α-oxoaldehyde Function Using Trifluoroacetyl-serine

This example describes the coupling of an oligonucleotide with a serine derivative, in order to introduce an α-oxoaldehyde function at the end of said oligonucleotide. In general, a threonine derivative may also be suitable, as may any β-amino alcohol carrying a carbonyl function in the α position.

a) Synthesis of trifluoroacetyl-serine

Synthesis of CF₃-CO-Ser(tBu)-OtBu

3 g of H-Ser(tBu)-OtBu (13.8 mmol, 1 eq) are dissolved in 50 ml of DCM (dichloromethane) freshly distilled over calcium hydride, in a 250 ml round-bottomed flask, with stirring and at 0° C. 15 ml of pyridine freshly distilled over calcium hydride (0.185 mmol, 13 eq) are added, followed by 4.3 ml of trifluoroacetic anhydride (4.3 ml, 2.2 eq). After 15 minutes, the reaction crude is concentrated under reduced pressure and is then taken up with 50 ml of ethyl acetate. The organic phase obtained is washed with a saturated solution of NaCl (twice 40 ml) and then dried over anhydrous sodium sulfate, filtered and concentrated under vacuum. The reaction crude is then taken up with toluene (30 ml) and then washed with a saturated solution of copper sulfate (3 times 30 ml) and again with a saturated solution of NaCl (twice 40 ml). The organic phase is then dried over anhydrous sodium sulfate, filtered and then concentrated under vacuum. A yellow oil is thus obtained (4.03 g, 93% yield).

Rf=0.47 (AcOEt/hexane 1/9); ¹H NMR (300 MHz, DMSO) δ(ppm): 1.2 (s, 9H, tBu), 1.5 (s, 9H, tBu), 3.5-4 (m, 2H, CH₂β), 4.5 (m, 1H, CHα).

Synthesis of CF₃-CO-Ser-OH

CF₃-CO-Ser(tBu)-OtBu (2 g, 6.39 mmol) is introduced into a 100 ml round-bottomed flask, followed by 10 ml of a trifluoroacetic anhydride (TFA)/water (7.5/2.5) mixture. After three hours, 10 ml of TFA are added. After 2 hours of further deprotection, the TFA and the water are concentrated under reduced pressure.

Rf=0.46 (CH₂Cl₂/MeOH/AcOH 77.5/15/7.5); ¹H NMR (300 MHz, DMSO) δ(ppm): 3.8 (m, 2H, CH₂β), 4.2 (m, 2H, CHα), 9.5 (unresolved peak, 1H, NH).

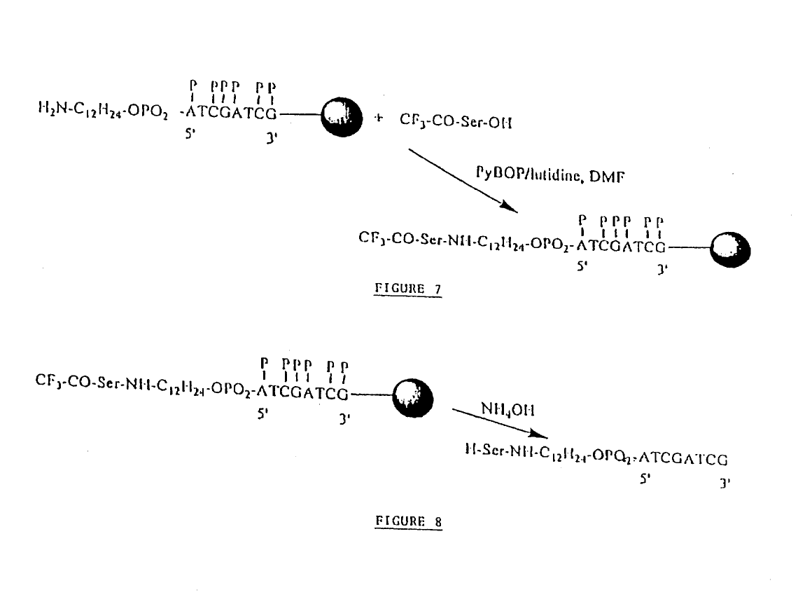

b) Coupling of the trifluoroacetyl-serine to the oligonucleotide (FIG. 7)

The coupling was carried out on batches of 1 μmol of oligonucleotide. The primary amine function of the aminolink in 5′ is kept protected with the monomethoxytrityl (MMT) protective group and deprotected at the last minute, just before the coupling with the trifluoroacetyl-serine, as described above.

Two solutions are prepared independently; namely, tube A: 49.5 μl of lutidine (85 eq), 46 mg of CF₃-CO-Ser-OH (46 eq) solubilized in 0.5 ml of DMF, and tube B: 120 mg of PyBop (46 eq) solubilized in 0.5 ml of DMF (dimethylformamide).

The support containing the oligonucleotide is pre-conditioned for 3 minutes with a solution of 49.5 μl of lutidine in 1 ml of DMF. After having removed the conditioning solution by filtration, the contents of tube A and then tube B are drawn up into the syringe, which is then agitated manually for 5 minutes. After the reagents have been removed by filtration, the resin is washed with DMF (3 times 2 minutes) and with DCM (twice 2 minutes), and is then dried with argon.

In FIG. 7, the nucleotide-protecting groups P are benzoyl for bases A and C and isobutyryl for base G.

c) Aminolysis (FIG. 8)

The resin is mixed together with 250 μl of 32% NH₄OH for 15 minutes. The aminolysis solution is recovered. This operation is repeated 3 times. The ammoniacal solutions obtained, and also 1 ml of solution for rinsing the support with 32% aqueous ammonia, are transferred into a clean and pyrolyzed glass tube. The tube is hermetically closed and left to stir at 60° C. overnight. The following day, the tube is cooled in an ice bath. The oligonucleotide solution is transferred into a hemolysis tube. The glass tube is rinsed with 1 ml of water and this aqueous solution is poured into the same hemolysis tube as previously. The solution is then evaporated under reduced pressure.

d) Quantification, analyses and purification by RP-HPLC

The crude residue is taken up in 2,500 μl of water. Assaying at 260 nm gives 2347.95 μg of crude oligonucleotide. Analysis of the crude by RP-HPLC: the crude reaction medium is purified on a C18 hyperprep column (30° C., detection at 260 nm, buffer A: 99/1 by volume 100 mM TEAA, pH=6.5/CH₃CN, buffer B: 95/5 by volume CH₃CN/H₂O, gradient: 0 to 40% of B in 86 minutes, flow rate: 3 ml/min). The fractions corresponding to the product are frozen and lyophilized.

e) Quantification, analyses by RP-HPLC and electrospray mass spectrometry

The residue is taken up in 1 ml of water. Assaying at 260 nm gives 175.3 μg of oligonucleotide. Analysis by RP-HPLC: 35 μl of the solution are diluted with 25 μl of water. 30 μl of these solutions are then injected onto a 250×4.6 mm C18 hypersil column under the same analytical conditions as above. The product is 99% pure. Analysis by electrospray mass spectrometry: buffer comprising 20% i-PrOH in H₂O is infused continuously. 10 μl of the oligonucleotides in solution in H₂O/20% i-PrOH/1% TEA are injected at a concentration of 10 pmol/μl. Analysis is carried out in negative ionization mode and the voltage cone is 50V. The flow rate is 10 μl/min and the temperature is 70° C. The observed molar mass for the product of the following formula is 2758.0:

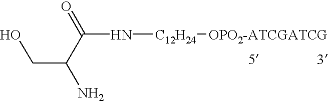

f) Periodate oxidation (FIG. 9)

46.1 μg of oligonucleotide are taken up in 25.6 μl of 100 mM phosphate buffer, pH=6.92. 0.98 mg of NaIO₄ are dissolved in 416.3 μl of water. 2.56 μl of this solution (i.e. 6.03 μg of NaIO₄) are removed and are deposited on the oligonucleotide in solution. 35 minutes later, the excess NaIO₄ is consumed with 2 eq of tartaric acid relative to NaIO₄, i.e. 2.82 μl of a solution containing 0.81 mg of tartaric acid in 270 μl of water. The reaction medium is stirred for a few minutes and 4 μl of reaction medium are removed in order to perform RP-HPLC. For this, 4 μl of reaction medium are removed and diluted with 96 μl of water. RP-HPLC is thus performed on a 250×4.6 mm C18 hypersil column (30° C., detection at 260 nm, buffer A: 99/1 by volume 100 mM TEAA, pH=6.5/CH₃CN, buffer B: 95/5 by volume CH₃CN/mQ H₂O, gradient 1 to 40% of B in 28 minutes, injected volume 70 μl, flow rate 1 ml/min). The reaction is complete.

g) Purification of the oxidized product by RP-HPLC

The reaction medium is injected onto a C18 hyperprep column (30° C., detection at 260 nm, buffer A: 99/1 by volume 100 mM TEAA, pH=6.5/CH₃CN, buffer B: 95/5 by volume CH₃CN/H₂O, gradient 0 to 40% of B in 86 minutes, flow rate 3 ml/min). The fractions corresponding to the product are pooled, frozen and lyophilized.

h) Quantification and electrospray mass spectrometry

The residue is taken up in 1,000 μl of water. Assaying at 260 nm gives 19.87 μg of oligonucleotide. ES-MS: the product is analyzed under conventional conditions. The observed mass is 2728.0.

### EXAMPLE 2

### Other Example of Production, by Solid-phase Chemistry, of Oligonucleotides Modified in the 5′ Position by an α-oxoaldehyde Function

1) Synthesis of Oligonucleotides

In this example, the oligonucleotides have sequences which are longer than those of the oligonucleotides according to the previous example. These oligonucleotides will be called, in the following text, primers 1 and 2, and have, respectively, the following sequences:

Primer 1: H₂N—C₆H₁₂-spacer arm-GTC CAA GCT CAG CTA ATT

Primer 2: H₂N—C₆H₁₂-spacer arm-GCA GGA CTC TAG AGG ATC

As described in the previous example, the primary amine function of the aminolink in the 5′ position of the oligonucleotide is kept protected with the MMT protective group and deprotected at the last minute, before coupling with the tartaric anhydride. This reaction is represented diagrammatically in FIG. 10, which represents primer 1, in which the nucleotide-protecting groups P are benzoyl for bases A and C and isobutyryl for base G. For primers 1 and 2, the spacer arm has the following formula:

—OPO₂—(OCH₂CH₂)₆OPO₂—(OCH₂CH₂)₆—OPO₂—

2) Modification of Primers 1 and 2 in the 5′ Position by an α-oxoaldehyde Function

a) Coupling of the primers with (+)-diacetyl-L-tartaric anhydride (FIG. 11, in which primer 1 is represented), and then aminolysis reaction (FIG. 12, in which primer 1 is represented)

The procedure is carried out as described in example 1.

b) Analyses and purification by RP-HPLC

For each one of the primers, 99 μl of water are added to 1 μl of the stock solution. 70 μl of this solution are then injected onto a 250×4.6 mm C18 hypersil column (30° C., detection 260 nm, buffer A: 99/1 by volume 100 mM TEAA, pH=6.5/CH₃CN, buffer B: 95/5 by volume CH₃CN/H₂O, gradient 1 to 100% of B in 65 minutes for primer 1, gradient 1 to 40% of B in 28 minutes for primer 2, flow rate 1 ml/min). Primer 1 is 72.1% pure, primer 2 is 59.5% pure. The two primers are lyophilized again and then taken up in 2,000 μl and 3,000 μl of water, respectively. These solutions are purified on a C18 hyperprep column (30° C., detection 260 nm, buffer A: 99/1 by volume 100 mM TEAA, pH=6.5/CH₃CN, buffer B: 95/5 by volume CH₃CN/H₂O, gradient 0 to 40% of B in 86 minutes, flow rate 3 ml/min). The fractions corresponding to the product are frozen and lyophilized.

c) Quantification, analyses by RP-HPLC and electrospray mass spectrometry

The primers are each taken up in 1000 μl of water.

Assaying at 260 nm gives 1605.12 μg of primer 1 and 1771.44 μg of primer 2.

Analysis by RP-HPLC: for each of the primers, 60 μl of water are added to 5 μl of the stock solution. 35 μl of this solution are then injected onto a 250×4.6 mm C18 hypersil column under the same analytical conditions as above. Primer 1: purity>99%; primer 2: purity of 98.6%.

Analysis by electrospray mass spectrometry: buffer comprising 20% i-PrOH in H₂O is infused continuously. 10 μl of the primers in solution in H₂O/20% i-PrOH/1% TEA are injected at a concentration of 10 pmol/μl. The analysis is carried out in negative ionization mode and the voltage cone is 50V. The flow rate is 10 μl/min and the temeprature is 70° C.

Primer 1, represented below: expected M 6458.48, obtained M 6454.5.

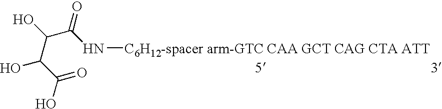

Primer 2, represented below: expected M 6548.533, obtained M 6544.0.

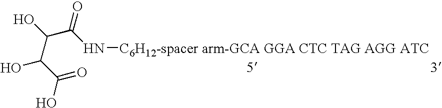

d) Periodate oxidation (FIG. 13, in which primer 1 is represented)

The procedure is carried out as described in example 1, with the difference that the oxidation reaction is carried out at a pH of 6.56. The concentration of NaIO₄ is 1 mM in the case of primer 1, and 1 mM or 5 mM in the case of primer 2 (separated into two batches).

e) Purification of the oxidized product by RP-HPLC

The reaction medium is diluted with water and injected onto a C18 hyperprep column under the same conditions as above. In each case, the product is frozen and lyophilized.

f) Quantification, RP-HPLC analysis and analysis by mass spectrometry

The residues are taken up in 1,000 μl of water. Assaying at 260 nm gives the following results: 785.4 μg of primer 1, 500.3 μg of primer 2 (for the batch oxidized with 1 mM of NaIO₄) and 521.4 μg of primer 2 (for the batch oxidized with 5 mM of NaIO₄).

For each of the three batches of primers (primer 1, primer 2 oxidized with 1 mM of NaIO₄ and primer 2 oxidized with 5 mM of NaIO₄), 53 μl, 49 μl and 50 μl, respectively, of water are added to 7 μl, 11 μl and 10 μl, respectively, of stock solution. 30 μl of these solutions are then injected onto a 250×4.6 mm C18 hypersil column (30° C., detection 260 nm, buffer A: 99/1 by volume 100 mM TEAA, pH=6.5/CH₃CN, buffer B: 95/5 by volume CH₃CN/H₂O, gradient 1 to 40% of B in 28 minutes, flow rate 1 ml/min). The purity of the primers in each of the batches is 81.6%, 90.4% and 95.2%, respectively.

Analysis by electrospray mass spectrometry: the analyses are carried out under the same conditions as above. Primer 1: M+H₂O (hydrate) expected 6400.4, observed 6397.0. Primer 2: M+H₂O (hydrate) expected 6490.4, observed 6487.0.

g) Counter-ion exchange

The triethylammonium counter-ions originating from the RP-HPLCs were exchanged against ammonium ions. This is carried out on a small exchange column filled with AG 50W-x8 resin, 200-400 mesh, from BIO-RAD, the H⁺ ions of which were exchanged beforehand against NH₄⁺ ions. The primers are deposited in water and also eluted with water. The primers are collected directly in receptacles containing penicillin, the column being connected to a UV detector. The solutions are frozen and lyophilized.

h) Quantification and RP-HPLC analysis

The primers are each taken up in 1,000 μl of water. Assaying at 260 nm gives the following results.

Primer 1 (1 mM of NaIO₄): 781.4 μg of oligonucleotide.

Primer 2 (1 mM of NaIO₄): 500.9 μg of oligonucleotide.

Primer 2 (5 mM of NaIO₄): 505.6 μg of oligonucleotide.

For the RP-HPLC analysis, the procedure is carried out as in f) above. The following percentages of purity are obtained: 80.2% for primer 1, 91.6% for primer 2 (1 mM of NaIO₄) and 92.7% for primer 2 (5 mM of NaIO₄).

### EXAMPLE 3

### Production, by Homogeneous Phase Chemistry, of Oligonucleotides Modified in the 5′ Position by an α-oxoaldehyde Function

1) Coupling of the Oligonucleotide with Disuccinimidyl Tartrate (FIG. 14)

As in example 1, the oligonucleotide used in the present example has the following sequence: ATCGATCG. It is provided by Genset Paris (reference: L00032077, oligo No. 1289153). It is taken up in 1 ml of deionized water and assayed (19.8 OD in 1,000 μl of water). 100 μl (65.34 μg of oligonucleotide) of the stock solution are then evaporated under reduced pressure. The residue is taken up in 100 μl of a 100 mM sodium bicarbonate solution, pH=8.51. After stirring, 1 mg (115 eq) of disuccinimidyl tartrate, dissolved in 100 μl of THF stabilized with 0.025% of BHT, are added to the oligonucleotide in solution.

The reaction is monitored by injecting a sample of the reaction medium onto an ion exchange column. For this, 5 μl of the reaction medium are removed, diluted with 65 μl of water and injected onto a Nucleogen ET 125/4 DEAE 60/7 column (50° C., detection 260 nm, buffer A: 80/20 by volume 20 mM KH₂PO₄/K₂HPO₄, pH=7.21/CH₃CN, buffer B: 80/20 by volume 20 mM KH₂PO₄/K₂HPO₄ 1M KCl, pH=6.67/CH₃CN, gradient 0 to 30% of B in 5 minutes and then 30 to 100% of B in 42 minutes, volume injected 70 μl, flow rate 1 ml/min).

After 6 hours, the reaction is terminated. 2 μl of 32% NH₄OH are added to the reaction medium to consume the excess N-hydroxysuccinimide ester. After 24 hours, the reaction medium is taken up with 2210 μl of water and purified on the same ion exchange column using the same elution conditions. The product is frozen and lyophilized.

2) Desalting

The residue is taken up in 200 μl of desalting buffer A (5 mM TEAA, pH=7.04). A 10 ml syringe is placed above a desalting cartridge (Sep Pak Plus C18 cartridge). 7 ml of 95% CH₃OH in water are passed through this, followed by 3 times 7 ml of buffer A. The 200 μl of the oligonucleotide solution are then loaded onto the cartridge. The salts are eluted with 7 ml of buffer A. The oligonucleotide is finally eluted with 3 times 3 ml of buffer B (50/50 5 mM TEAA, pH=7.04/CH₃OH) and the elution is monitored by UV assay at 260 nm. The fraction containing the oligonucleotide is concentrated under reduced pressure.

3) Quantification, Analyses by Ion Exchange and Mass Spectrometry

The residue is taken up in 1 ml of water. Assaying at 260 nm gives 15.32 μg of oligonucleotide. Analysis by electrospray mass spectrometry: 5 μl of the oligonucleotide in solution in H₂O/20% i-PrOH/1% TEA are injected at a concentration of 2.8 pmol/μl. The analysis is always carried out under the same conditions as above. An oligonucleotide is obtained, corresponding to the following formula, with M 2720.0; (M−H+Na) 2742.0; (M−H+K) 2757.0:

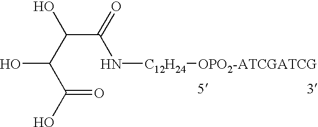

4) Periodate Oxidation

The procedure is carried out as described in examples 1 and 2.

### EXAMPLE 4

### Production of Oligonucleotides Modified in the 3′ Position by an α-oxoaldehyde Function

Oligonucleotides modified in the 3′ position by an α-oxoaldehyde function, which can be immobilized on a suitably functionalized support so as to prepare a biochip in accordance with the present invention, can be obtained using the functionalized solid support described in the international PCT application published under the number WO 00/64843 and in the article by J. S. FRUCHART et al., published in Tetrahedron Letters, 1999, 40, 6225-6228.

1) Preparation of a Functionalized Solid Support (FIG. 18)

Coupling propanolamine to diacetyltartaric anhydride

1.297 g of (+)-diacetyl-L-tartaric anhydride (6 mmol) are solubilized in 2 ml of DMF, in a 10 ml round-bottomed flask at 0° C. with stirring and under argon pressure. 0.459 ml of propanolamine (6 mmol) are added, followed by 0.843 ml of triethylamine (6 mmol). The reaction mixture is left to stir for 5 minutes.

Coupling 2,3-diacetoxy-N-(3-hydroxypropyl)-succinamic acid to an Argogel®-NH₂ resin

263.18 mg (0.1 mmol) of Argogel®-NH₂ resin carrying a load of 0.38 mmol/g are conditioned, in a syringe, with 3 successive washes of 3 minutes in DMF. 1.6 ml of the solution of 2,3-diacetoxy-N-(3-hydroxypropyl)succinamic acid synthesized above are added, followed by 1.56 g of PyBOP (3 mmol) solubilized in 1 ml of DMF. After stirring for 15 minutes, the excess of reagent is removed by filtration and the resin is then washed by successive washes with DMF (3 times 3 minutes) and with DCM (twice 3 minutes), before being dried under vacuum. A positive Kaiser test shows the absence of free amine on the resin at the end of the reaction.

HR-MAS NMR (with diffusion filter) (δ ppm): 1.7 (s, 2H, CH₂CH₂CH₂), 2.1 (s, 6H, 2×CH₃CO), 4.2 (m, 2H, CH₂OH), 5.7 (s, 2H, 2×CH tartrate), 6.5-7.2 (m, PS resin), 7.9 (m, 1H, NH), 8.2 (m, 1H, NH).

It is clearly understood that, as a variant, solid supports other than Argogel®-NH₂ resin may be used, such as CPG supports (controlled pore glass beads).

Protection of the free hydroxyl function with DMT

The previously functionalized resin is conditioned with 3 successive washes of 3 minutes in pyridine. 1 g of 4,4′-dimethoxytrityl chloride (3 mmol) solubilized in 5 ml of pyridine is added. After stirring for 1 hour, the excess of reagents is removed by filtration and the resin is then washed by successive washing with pyridine (5 times 3 minutes) and with 3% triethylamine in DCM (3 times 3 minutes), before being dried under vacuum. HR-MAS NMR (with diffusion filter) (δ ppm): 1.8 (s, 6H, 2×CH₃CO), 2-2.5 (m, 2H, CH₂CH₂CH₂), 3-3.6 (m, PEG resin), 3.7 (s, 6H, —O—CH₃ DMT), 5.4 (s, 2H, 2*CH tartrate), 6.5-7.2 (m, PS resin and aromatics of DMT), 7.7 (m, 1H, NH), 8.3 (m, 1H, NH).

2) Production of an Oligonucleotide Carrying an α-oxoaldehyde Function in the 3′ Position, Using the Solid Support Obtained Above

(T)₆—PO₂—O—(CH₂)₃—NH—CO—CHO is synthesized as detailed below.

After the oligonucleotide synthesis on the solid support prepared in 1) above, in accordance with methods known to those skilled in the art, 1 μmol of oligonucleotide deprotected in 5′ (removal of the DMT group) is introduced into an eppendorf equipped with a pressure-resistant seal, in the presence of 1 ml of aqueous ammonia at 32% in water. After leaving this overnight at 60° C., the reaction mixture is cooled and is then transferred into a syringe. The ammoniacal solution is removed by filtration, and the resin is then washed by successive washes with 32% aqueous ammonia (twice 3 minutes) and then with water, until the washing solution is neutral, as determined by measuring with pH paper. The resin is then dried under vacuum.

Periodate oxidation is then carried out via the following steps. The previously functionalized resin is conditioned with 3 successive washes of 3 minutes in a 0.1 M phosphate buffer at pH=6.4. One tenth of a solution consisting of 36.3 mg (170 μmol) of sodium periodate solubilized in a mixture of water (0.1 ml) and 0.1 M phosphate buffer at pH=6.4 (5 ml) is added. After stirring for 1 hour, 5.1 mg of tartaric acid (34 μmol) solubilized in 500 μl of water are added. After 2 minutes, the oxidation solution is recovered in a tube by filtration. The resin is washed by 2 successive washes with water, the filtration water being combined with the previous oxidation solution (total volume=3.63 ml).

The oxidized product is purified by RP-HPLC and lyophilized. For the RP-HPLC, the reaction medium is directly injected onto a C18 hyperprep column (t°=30° C., 260 nm, buffer A=99/1 100 mM TEAA, pH=6.5/CH₃CN, buffer B=95/5 CH₃CN/H₂O, gradient=0 to 40% of B in 86 minutes, volume injected=3.53 ml, flow rate=3 ml/min). The major product is isolated: 213.93 μg of oligonucleotides (11% yield) by quantitative measurement of the OD. Lyophilization in the presence of 1426.9 μg of mannitol and 0.189 μl of tri-N-butyltriphenylphosphine.

### EXAMPLE 5

### Preparation of Glass Surfaces Functionalized with a Hydrazide Group (FIG. 15)

In order to be able to attach to the nucleic acids carrying an α-oxoaldehyde function at their 3′ or 5′ end, in accordance with the method according to the invention, the surface of the glass slides needs to be suitably adapted beforehand. In particular, the presence of a spacer arm may be useful to distance the probe from the surface and obtain optimal hybridization, and also to control, in part, the physicochemical properties of the surface (hydrophilicity, hydrophobicity, charge). The surface may also be adapted so as to increase the number of functional sites per unit of surface, for example by synthesizing dendrimeric structures on the glass (M. BEIER, et al., Nucleic Acids Research, 1999, 27, 1970-1977) or by coupling polyamines. The strategy for synthesis envisioned is represented in FIG. 15.

In order to determine the quality of the hydrazide slides synthesized, the same reaction as that which will be used to fix to oligonucleotides is used, i.e. a reaction of ligation with a compound functionalized with an α-oxoaldehyde group (cf. FIG. 16). A probe which makes it possible to characterize the slides with great sensitivity was chosen: it is a fluorescent peptide functionalized with an α-oxoaldehyde group.

1) Synthesis of a Fluorescent Probe Functionalized with an α-oxoaldehyde Group

A rhodamine peptide functionalized with an α-oxoaldehyde group of sequence (5)-6-carboxy-tetramethylrhodamine-Lys-Arg-NH—(CH₂)₃—NH—CO—CHO was synthesized using the support named IPT (2,3-O-isopropylidene-D-tartrate) described by J. S. FRUCHART et al. in Tet. Letters, 1999, 40, 6225-6228 and in the international PCT application published under the number WO 00/64843. The synthesis is summarized in FIG. 17. 500 mg of IPT resin (0.23 mmol/g) are used in a cycle of solid-phase synthesis according to a strategy of Fmoc/t-Bu in NMP (N-methylpyrrolidone) with the following reagents and amounts:

Fmoc-Arg(Pbf)-OH (0.298 g, 4 eq), HBTU (0.174 g, 4 eq), HOBt (62 mg, 4 eq), DIEA (240 μl, 12 eq) for 1 hour. NMP/piperidine (80/20) deprotection for 30 minutes,

Fmoc-Lys(Boc)-OH (0.125 g, 4 eq), HBTU (0.174 g, 4 eq), HOBt (62 mg, 4 eq), DIEA (240 μl, 12 eq) for 1 hour. NMP/piperidine (80/20) deprotection for 30 minutes,

(5)-6-carboxytetramethylrhodamine (99 mg, 2 eq), HBTU (0.087 g, 2 eq), HOBt (31 mg, 2 eq), DIEA (120 μl, 6 eq) for 1 hour.

The resin is washed with NMP (2×2 min) and then with DCM (2×2 min). The protections on the side chains and the isopropylidene group are deprotected with 5 ml of TFA in the presence of scavengers (375 mg of phenol, 125 mg of ethanedithiol, 250 μl of thioanisole and 250 μl of water). The resin is then conditioned in 5 ml of 33% acetic acid for 2 minutes. The peptide is then cleaved from the support by adding sodium periodate (0.147 g, 6 eq) diluted in 1 ml of water, with stirring, for 5 minutes. The resin is filtered and then washed with 10 ml of water (3 times 1 minute). The cleavage solutions are combined and added to 21 μl of ethanolamine (3 eq) before being purified immediately on a C18 RP-HPLC Hyperprep column (15×300 mm). 8 mg of peptide are obtained.

After the rhodaminated peptides have been fixed to the hydrazide slides, the slides will be washed in order to eliminate noncovalent adsorption, according to the following various protocols. Protocol 1: the slides are immersed in a solution of K₂HPO₄ at 5% in water, for 2 hours with ultrasound. The slides are rinsed successively with baths of 3 minutes in water (twice) and, finally, in methanol (once). The slides are then dried in a desiccator under vacuum. Protocol 2: the slides are washed with a 100 mM tris(hydroxymethyl)aminomethane acetate buffer, pH 5.5, containing 0.1% by mass of Tween 20, for 15 min.

2) Functionalization of the Glass Slides with a Hydrazide Group (Hydrazine-derived Function)

a) Steps of washing, stripping and silanizing commercial slides

Precleaned microscope slides (Esco) with ground edges and a frosted end are immersed in a bath of sodium hydroxide at 10% in water, first with ultrasound for 10 minutes and then overnight without ultrasound. After having rinsed these slides with three successive baths of 3 minutes in water, they are immersed in a solution of hydrochloric acid at 3.7% in water, for 4 hours. Prerinses of three minutes are performed with water (3 times) and then with methanol (once), before immersing the slides in a bath containing 3% aminopropyltrimethoxysilane in 95% methanol for 30 minutes with ultrasound. The slides are rinsed successively with baths of 3 minutes in methanol (once), water (twice) and, finally, methanol (once). The slides are then drained for a few minutes, dried for 15 minutes in an incubator at 110° C. and then stored in a desiccator under vacuum.

b) Functionalization of the silanized slides by reaction of a hydrazine derivative on an isocyanate

Formation of isocyanate

For the formation of isocyanate, triphosgene and carbonyldiimidazole were tested. However, many other reagents may be used. Various solvents were also tested: dichloromethane, DMF, tBu-OMe, toluene and dichloroethane. The previously silanized slides are immersed in a solution of dichloroethane containing triphosgene (100 mmol/l) and DIEA (800 mmol/l), for 2 hours. These slides are then rapidly drained before being directly immersed in the solution containing the hydrazine derivative.

Reaction of isocyanate with hydrazine or a derivative

Synthesis of Fmoc-NH—NH₂: 10 ml of hydrazine hydrate are introduced into a 1 liter round-bottomed flask, with stirring. 1 g of Fmoc-Cl dissolved in 250 ml of acetonitrile are added via a dropping funnel. The reaction mixture is stirred for 30 minutes at ambient temperature and is then concentrated under vacuum. The crude obtained is recrystallized from 200 ml of absolute ethanol and is then filtered on a sintered glass funnel. The white crystals are washed with absolute ethanol and then dried under vacuum (m=0.65 g, yield=65%). Rf=0.74 (CH₂Cl₂/TEA 9.5/0.5); T°f=162-165° C.; ¹H NMR (300 MHz, DMSO) δ (ppm): 4 (unresolved peak, 2H, NH₂), 4-4.1 (m, 3H, CH₂ and CH (Fmoc)); 7.2-7.9 (m, 8H (Fmoc)), 8.3 (unresolved peak, 1H, NH).

The slides functionalized with an isocyanate group are immersed in a solution of Fmoc-NH—NH₂ (22 mmol/l) in DMF for 2 hours with ultrasound. The slides are then rinsed successively with baths of 3 minutes in DMF (once), water (twice) and, finally, methanol (once), before being dried and stored in a desiccator under vacuum. Alternatively, the slides functionalized with an isocyanate group may be reacted with hydrazine (1% by volume) in DMF.

Deprotection

The method corresponding to the best conditions tested is as follows: the slides obtained above are immersed in a solution of DMF containing piperidine (0.2% by volume) and diazabicyclo undecene (DBU, 2% by volume) for 30 minutes. The slides are then rinsed successively with baths of 3 minutes in DMF (once), water (twice) and, finally, methanol (once), before being dried and stored in a desiccator under vacuum. Other deprotection systems may consist, for example, of DMF/piperidine (80/20) or DMF/piperidine/DBU (96/2/2) mixtures, the times of contact with the slides then being, respectively, 30 minutes or between 2 and 30 minutes.

3) Developing, Controlling the Quality of the Slides

The slides obtained are developed (quality control thereof) with the α-oxoaldehyde rhodaminated peptide synthesized above: the slides functionalized with a hydrazide group are soaked for 1 hour at 37° C. in a bath of the rhodaminated peptide functionalized with an α-oxoaldehyde group (64 μmol/l), in the presence of acetate buffer (100 mM, pH=5.5). The peptide not fixed covalently, but adsorbed onto the slide, is removed by soaking in a solution of K₂HPO₄ at 5% in water for 2 minutes, with ultrasound. The slides are rinsed successively with baths of 3 minutes of water (twice) and of methanol (once). The slides are then dried in a desiccator under vacuum and then passed through a scanner. The same experiment may be carried out with a rhodaminated peptide not carrying an α-oxoaldehyde function (negative control). Sequence of the peptide: rhodamine-Lys-Arg-NH₂.

### EXAMPLE 6

### Preparation of Glass Surfaces Functionalized with a Hydrazine, Hydroxylamine or β-aminothiol Group

The methods described below make it possible to produce glass surfaces suitably functionalized for the purpose of fixing nucleic acids modified in the 5′ or 3′ position by an α-oxoaldehyde function.

a) Silanization of glass slides

The glass slides are silanized as described by M. BEIER, et al. in Nucleic Acids Research, 1999, 27, 1970-1977 and by N. L. BURNS, et al. in Langmuir, 1995, 11, 2768-2776. The slides are treated overnight with an aqueous solution of sodium hydroxide (10%), washed with water, with hydrochloric acid at 1% in water, again with water and, finally, with methanol. After sonication for 15 minutes in 95% methanol containing 3% by volume of aminopropyltrimethoxysilane, the slides are washed with methanol and then with water and dried under a stream of nitrogen. They are heated for 1 minute at 110° C. After cooling, they are stored under nitrogen.

b) Functionalization of glass slides

With a hydrazine function

Fmoc-hydrazinoacetic acid (Fmoc: 9-fluorenylmethoxycarbonyl) of formula Fmoc-NH—NH—CH₂—COOH is synthesized from ethyl α-hydrazinoacetate hydrochloride (ALDRICH) by saponification of the ester function in sodium hydroxide, followed by protection of the hydrazine function, according to the protocol described by E. ATHERTON in The Peptides, 1987, 9, part C, S. Udenfriend and J. Meienhofer J. Eds., Academic Press, San Diego, Calif. The silanized glass slides are brought into contact with the Fmoc-hydrazinoacetic acid (100 mM) in the presence of BOP (100 mM) and of DIEA (diisopropylethylamine; 200 mM) in dimethylformamide (DMF), for 1 hour. These slides are then washed with DMF, treated with piperidine at 20% by volume in DMF for 5 minutes (removal of hydrazine function-protecting Fmoc groups) then washed with DMF and with methanol, and dried under nitrogen.

With a hydroxylamine function

The silanized glass slides are treated with Fmoc-aminooxyacetic acid of formula Fmoc-NH—O—CH₂—COOH (SENN CHEMICALS; 100 mM) in the presence of BOP (100 mM) and of DIEA (200 mM) in DMF, for 1 hour. They are washed with DMF and treated, for 5 minutes, with piperidine at 20% by volume in DMF (removal of hydroxylamine function-protecting Fmoc groups). They are then washed with DMF and with methanol and dried under nitrogen.

With a β-aminothiol function

The silanized glass slides are treated, in the presence of BOP (100 mM), of DIEA (200 mM) and in DMF, for 1 hour, with Fmoc-Cys(StBu)-OH acid (acid of formula Fmoc-NH—CH (CH₂SStBu)—COOH, corresponding to α-amino-β-mercaptopropionic acid, the thiol and amine functions of which are, respectively, protected with StBu and Fmoc groups; NOVABIOCHEM, 100 mM). After washing with DMF, they are treated with 20% piperidine in DMF for 5 minutes (removal of amine function-protecting Fmoc groups). They are then washed with DMF and with methanol, and treated with an aqueous solution of 100 mM tris(carboxyethyl)phosphine (TCEP) hydrochloride in a phosphate buffer, pH 7.0, for 30 minutes (removal of thiol group-protecting StBu groups). They are then washed with DMF and with methanol and dried under nitrogen.

### EXAMPLE 7

### Ligation of Nucleic Acids Functionalized with an α-oxoaldehyde Group to a Support Functionalized with Hydrazide Groups, to Produce DNA or Oligonucleotide Chips in Accordance with the Present Invention

The ligation of nucleic acids functionalized in the 5′ position with an α-oxoaldehyde function, onto a glass slide functionalized with a hydrazide function, is described below. The attachment of the nucleic acids to the glass slide results in the formation of hydrazone bonds (semicarbazone linkages). The efficiency of the ligation onto these slides was evaluated by hybridization of complementary oligonucleotides labeled in 5′ with a cyanine-3 (Cy-3) molecule. The same ligation experiment was carried out successfully using oligonucleotides functionalized in the 3′ position with an α-oxoaldehyde function, and mixtures of oligonucleotides functionalized in the 3′ or 5′ position.

1) Materials and Methods

Materials for deposition and for reading

The oligonucleotides were deposited onto glass slides using an Affymetrix® 417 Arrayer robot (Affymetrix Inc., 3380 Central Exwy, Santa Clara, Calif. 95051) equipped with a “pin and ring” (4 pins) sampling head. The pins have a diameter of 125 μm and make a circle-shaped deposit of approximately 150-170 μm in diameter for a volume of approximately 30-50 pl (volume stated by the supplier). The deposits were 375 μm apart from center to center. Detection of the fluorescent hybridization probe is obtained using an Affymetrix® 418 Array scanner equipped with 2 laser diodes for reading at excitation wavelengths of 532 and 635 nm. The fluorescence emitted by the fluorochromes after excitation is detected using a photo multiplier tube (PMT). The result is obtained in the form of a 16-bit image file with a resolution of 10 μm/pixel. The computer analysis of the image files and the quantification of the fluorescence intensity were carried out using the “ScanAlyze” freeware developed by M. EISEN of Stanford University.

Reagents

The following commercial solutions are used: 20×SSC (3M NaCl, 0.3M Na citrate; quantum bioprobe, Quantum Biotechnologies Inc.), NaBH₄ (sigma), PBS (phosphate buffered saline; Gibco Life Technology).

The oligonucleotides carrying an α-oxoaldehyde function in the 5′ position are as obtained in example 2 above. The oligonucleotides used in the present ligation protocol correspond to the following formulae (“α-oxo” indicates the presence of an α-oxoaldehyde function):

P1-α-oxo=5′-α-oxo-GTC CAA GCT CAG CTA ATT-3′;

P2-α-oxo=5′-α-oxo-GCA GGA CTC TAG AGG ATC-3′;

P1-diol=5′-diol-GTC CAA GCT CAG CTA ATT-3′;

P1-tartrate=5′-tartrate-GTC CAA GCT CAG CTA ATT-3′.

The sequences of the Cy3-labeled complementary oligonucleotides are as follows:

Complementary P1-Cy3=5′-Cy3-AAT TAG CTG AGC TTG GAC-3′

Complementary P2-Cy3=5′-Cy3-GAT CCT CTA GAG TCC TGC-3′

The functionalized oligonucleotides are diluted in water and kept at −20° C. until use. The amount required for the deposits is taken from this stock and lyophilized before being resuspended in the depositing solution.

Depositing the oligonucleotides onto the glass slides

The glass slides used are functionalized with a hydrazide group and are as obtained in example 5 above. Different amounts of lyophilized oligonucleotides were resuspended in 20 μl of solution in order to obtain concentrations of 0.1 mM, 0.05 mM, 0.01 mM and 0.001 mM. Various resuspension solutions were tried in order to obtain the best possible spot shape. The deposits were made 375 μm apart from one another, at a temperature of 20° C. and in an atmosphere at 70% (±5%) relative humidity. After deposition, the slides were incubated in a humidity-saturated chamber (close to 100% relative humidity) at 37° C. for 14 to 16 h. The slides were then washed in a 0.1% SDS solution for 5 minutes at ambient temperature in order to remove the oligonucleotides which had not reacted with the slide. This washing step was optimized in order to eliminate a maximum of aspecific adsorption between the oligonucleotide and the glass. After washing, the slides are dried by centrifugation (5 minutes; 30×g; 20° C.) in the vertical position.

Prehybridization, hybridization and washing

The hybridizations are carried out in “CMT-hybridization chambers” (Corning). The deposit area is prehybridized with 15 μl of prehybridization buffer (50% formamide, 4×SSC, 0.5% SDS; 2.5×Denhardt's) at 50° C. for 1 h 30. The prehybridization solution is placed between slide and cover slip. The slide is placed in the hybridization chamber, which contains 2 reservoirs which received approximately 15 μl of prehybridization buffer in order to saturate the atmosphere inside the chamber with humidity. The chamber is hermetically closed and immersed in a water bath at 50° C.

After prehybridization, the chamber is opened, the cover slip is removed and the prehybridization buffer is discarded by inclining the slide on abosrbent paper. 15 μl of hybridization buffer (50% formamide; 6×SSC; 0.4% SDS; 4×Denhardt's; 0.01 mM of complementary oligonucleotide) are prepared and incubated at 95° C. for 5 min, before being placed on the deposit area. The slide is re-covered with the cover slip and placed in the hybridization chamber so as to be incubated at 50° C. for 14 to 16 h. This process should be carried out as rapidly as possible in order to avoid any drying after having removed the cover slip.

After hybridization, the slide is immersed in 50 ml of 2×SSC in the vertical position in order to detach the cover slip. After detachment, the slide is washed successively in 50 ml of 0.1% of SDS, 0.1×SSC for 5 min; 50 ml of 0.1×SSC for 5 min; 50 ml of 0.1×SSC for 5 min. These washes are carried out in 50 ml Falcon tubes, at room temperature. Agitation is effected by turning the tube over once every minute. After the final wash, the slides are rinsed under a jet of sterile water and dried immediately by centrifugation (5 minutes; 30×g; 20° C.).

Reading the slides on the scanner

The slides are scanned at a wavelength of 532 nm (Cy-3), immediately after washing. Reading is carried out at various settings for the laser power and for the aperture of the PMT tube. A standard setting (35% laser power, 50% PMT aperture) was chosen and used systematically for all the readings in order to be able to visually compare the results with one another. When quantification of the fluorescence intensity is desired, the L/PMT setting is modified so as to obtain all the fluorescent signals below the saturation threshold.

Synthesis of PCR products, depositions and hybridization

The P1-tartrate and P2 oligonucleotides were used in a PCR on the plasmid pFus II comprising a fragment of the bordetella pertussis S1 gene: pFus II+S1 (the portion amplified corresponds to the inserted gene fragment). The PCR was carried out using AmpliTaq Gold® from Perkin Elmer and under the conditions recommended by the supplier. The amplification cycles are as follows: 1×(94° C., 10 min); 35×(94° C., 45 sec/55° C., 45 sec/72° C., 45 sec); 1×(72° C., 10 min).

After PCR, the products obtained were distributed into 4 wells of a Multiscreen PCR plate (Millipore) and treated in the following way. The reaction liquid was filtered through the membrane by suction. The DNA fixed to the membrane was washed 3 times with 100 μl of 3×SSC buffer, pH 5.5. The DNA was resuspended in 50 μl of 6×SSC and the tartrate function was oxidized with 50 μl of sodium periodate (2 mM in water). The various wells were subjected to various oxidation times: 0 h (nonoxidized negative control), 30 min, 1 h and 3 h. After oxidation, the reaction was stopped with 100 μl of tartaric acid (in 3×SSC) for 10 min. The products were then washed 3 times with 3×SSC, before being taken up in water, evaporated and resuspended in the depositing solution (3×SSC). The concentrations obtained were 0.3 to 0.4 μg/μl. Two controls (oxidized and nonoxidized) were added to the experiment: they correspond to the same PCR amplification obtained with the pair of oligonucleotides P1-P2.

These PCR products were deposited and ligated under the same conditions as in 3) above. The hybridization was carried out as indicated in 4) above, using a probe synthesized by unidirectional PCR on the plasmid pFus II+S1 and using the oligonucleotide P2 to initiate synthesis. After hybridization, the slides were washed and analyzed as described in 4) and 5) above.

2) Results

Appearance of the deposit and aspecific adsorption

Various depositing solutions, at various concentrations and various pHs, were tested. The quality of the result was estimated by depositing the complementary oligonucleotide P1 directly at a concentration of 0.1 or 0.01 mM. The shape, the intensity and the homogeneity of the spot were evaluated visually and by quantifying the fluorescence intensity. The best results were obtained using 3×SSC, pH 5.5. Acceptable results were also obtained by making the deposits in a solution of 1 mM Tris acetate, pH 5.5+450 mM NaCl. These two solutions were used to deposit P1-α-oxo onto the hydrazide slides in order to quantify the rate of hybridization. The washing of oligonucleotides not fixed to the slide was optimized in order to obtain minimal aspecific adsorption. The best washing protocol makes it possible to remove 90% of unfixed oligonucleotides.

Hybridization on slides functionalized with a hydrazide group

The depositing of P1-α-oxo onto a hydrazide slide, and also the hybridizations with the complementary oligonucleotide P1, were carried out as detailed above. The results of the hybridization are given in tables 1 and 2. The deposits were made in 3×SSC, pH 5.5 (table 1), and in Tris acetate, pH 5.5+450 mM NaCl (table 2). The values of the fluorescence intensities given in tables 1 and 2 were measured using the ScanAlyze program. The laser and PMT settings were first modified so as to obtain an image exhibiting no saturation (30% laser and 35% PMT). The mean values from 6 replicates for each deposit, and a standard deviation for these values, are given in tables 1 and 2.

TABLE 1
Fluorescence intensity of oligonucleotides deposited, in a
3 × SSC buffer medium, pH 5.5, onto a hydrazide slide
and hybridized with Cy3-labeled complementary sequences

| | Type of oligonucleotide | | | | |
|---|---|---|---|---|---|
| | P1-α- | P1-α- | P1-α- | P1-α- | P1- |
| | oxo | oxo | oxo | oxo | diol |
| Oligonucleotide | 0.1 | 0.5 | 0.01 | 0.001 | 0.1 |
| concentration (mM) | | | | | |
| Mean of fluorescence | 10.871 | 8.497 | 1.496 | 162 | 441 |
| intensity | | | | | |
| Standard deviation | 692 | 634 | 72 | 8 | 74 |
| (on 6 measurements) | | | | | |

TABLE 2
Fluorescence intensity of oligonucleotides deposited, in a Tris
acetate pH 5.5 + 450 mM NaCl buffer medium, onto a hydrazide
slide and hybridized with Cy3-labeled complementary sequences

| | Type of oligonucleotide | | | | |
|---|---|---|---|---|---|
| | P1-α- | P1-α- | P1-α- | P1-α- | P1- |
| | oxo | oxo | oxo | oxo | diol |
| Oligonucleotide | 0.1 | 0.5 | 0.01 | 0.001 | 0.1 |
| concentration (mM) | | | | | |
| Mean of fluorescence | 2.856 | 2.061 | 977 | 164 | 228 |
| intensity | | | | | |
| Standard deviation | 214 | 240 | 88 | 15 | 20 |
| (on 6 measurements) | | | | | |

Considerable hybridization can be detected on the deposits (tables 1 and 2), whereas a nonhybridized slide has a very weak fluorescence (fluorescence intensity: 50-70). The strongest fluorescence intensity is obtained for a deposit at 0.1 mM of oligonucleotide P1-α-oxo and in the 3×SSC buffer, pH 5.5. The fluorescence intensity decreases with the concentration of oligonucleotide deposited. At 0.001 mM, the signal becomes barely detectable.

A control of aspecific adsorption is obtained by depositing an oligonucleotide which has the same nucleotide sequence as P1-α-oxo, but the functionalization of which is incomplete (arrest at diol step), and which does not thereby have the possibility of reacting with the semicarbazide functions of the support. This oligonucleotide is deposited at a concentration of 0.1 mM and exhibits a fluorescence intensity which is much weaker than the P1-α-oxo equivalent. The values summarized in table 1 make it possible to estimate that the intensity from aspecific fixing represents only approximately 4% of the signal (10 871 from 0.1 mM P1-α-oxo, compared to 441 for 0.1 mM P1-diol).

In general, the deposits in Tris acetate buffer (table 2) have the same characteristics as in 3×SSC (table 1), but with weaker fluorescence intensities. The protocols for depositing (in 3×SSC) and for hybridization detailed above were repeated several times under the same conditions, and identical results were obtained.

The same concentration range was prepared and deposited using an oligonucleotide with a different sequence: P2-α-oxo. The depositing and the fixing on a hydrazide slide were carried out under the same conditions as for P1-α-oxo. The hybridization was carried out using an oligonucleotide labeled with a Cy3 in 5′ and complementary to P2. After hybridization, the fluorescence measurements are highly comparable to those obtained with P1-α-oxo, proving that the results obtained with P1-α-oxo are verified with oligonucleotide pairs having different sequences.

Reuse of slides (dehybridization-rehybridization)

Tests comprising dehybridization and rehybridization were carried out on a deposit of 0.1 mM of P1-α-oxo on a hydrazide slide. After the first hybridization (with 0.01 mM of complementary oligonucleotide P1) and reading of the results, the slides were immersed in water at 95° C. for 5 minutes. The slides were then dried and the fluorescent signal still present on the deposits was evaluated by reading again on the scanner. After three successive hybridization/dehybridization cycles, the results obtained show that the DNA deposited was dehybridized almost completely and then rehybridized so as to attain a level of fluorescence comparable to the initial hybridization. These tests show that the oligonucleotides fixed to the hydrazide slides withstand the dehybridization conditions and remain accessible so as to undergo successive hybridizations.

Results of hybridization on PCR products

In this test, the P1-tartrate oligonucleotides are used in a PCR reaction. They are then subjected to a peroxidation reaction (reaction time: 30 minutes, 1 h or 3 h) and the oligonucleotides thus oxidized are deposited onto the hydrazide slide. An increase in the fluorescent signal is observed as a function of the oxidation time, showing that the tartrate function is correctly transformed into an α-oxoaldehyde function, and that there are therefore more and more PCR products available for the ligation.

3) Conclusion

The results presented in this example show that oligonucleotides functionalized in 5′ with an α-oxoaldehyde function may be fixed to a hydrazide slide, and that the oligonucleotides, once fixed, remain accessible for hybridization with complementary oligonucleotides. It is also possible to dehybridize, by heating to 95° C., the oligonucleotides fixed to the slide and to reuse this slide in a new hybridization. With regard to the oligonucleotide carrying, at its 5′ end, a tartrate group, it may be used in a PCR reaction and then oxidized before being deposited onto the hydrazide slide. Once fixed to the slide, the PCR product may be hybridized with a complementary nucleotide sequence.

### EXAMPLE 8

### Depositing Oligonucleotides Modified in the 5′ Position by an α-oxoaldehyde Function onto Glass Slides Functionalized with Hydrazine, Hydroxylamine or α-aminothiol Groups

a) In the case of glass slides functionalized with hydrazine or hydroxylamine groups

The oligonucleotides modified in the 5′ position by an α-oxoaldehyde function, as obtained in the previous examples, are taken up into solution in a phosphate buffer, pH 6.0, and then deposited manually or using a robot onto the glass slides obtained in example 6. The deposition is accompanied by immobilization of the oligonucleotides on the surface by formation of hydrazone bonds (when the surface carries a hydrazine function) or oxime bonds (when the surface carries a hydroxylamine function).

The slides are incubated in a humid chamber overnight at 37° C. They are washed with water and then subjected to “stripping” treatment with disodium phosphate (Na₂HPO₄; 2.5 mM) and 0.1%, by volume, of SDS (sodium salt of the dodecyl sulfate ester) at 95° C. and for 30 seconds. After washes with water, the slides are dried under a stream of nitrogen and stored under an inert atmosphere.

b) In the case of glass slides functionalized with β-aminothiol groups

The oligonucleotides modified in the 5′ position by an α-oxoaldehyde function, as obtained in the previous examples, are taken up in solution in a phosphate buffer, pH 6.0, containing 1 mM of TCEP (tris(carboxyethyl)phosphine hydrochloride), and then deposited manually or using a robot onto the glass slides obtained in example 6. Immobilization of the oligonucleotides on the glass slide is accompanied by the formation of thiazolidine bonds. The slides are incubated and treated as described in a).

The protocols described in a) and b) above may also use oligonucleotides modified in the 3′ position by an α-oxoaldehyde function, or longer nucleic acids such as DNAs.

### EXAMPLE 9

### Ligation between a Peptide Having a Hydrazine Function in the N-terminal Position and an Oligonucleotide Having an α-oxoaldehyde Function in the 5′ Position

This example illustrates the ligation of an oligonucleotide in accordance with the invention to a nonsolid support which is peptide in nature.

1) Synthesis of the Peptide of Formula H₂N-GRYL-NH₂

The peptide synthesis was carried out according to the Fmoc/t-Bu strategy on an Applied Biosystems 431A synthesizer, on 0.25 mmol of Rink Amide MBHA resin® carrying a load of 0.74 mmol/g. The amino acids are activated using an HBTU/HOBt/DIEA mixture (amino acid/HBTU/HOBt/DIEA: 4 eq/4 eq/4 eq/8 eq) in NMP. The side chains are protected as follows: Arg(Pbf), Tyr(t-Bu).

At the end of synthesis, the resin is divided into 2 batches. On one half (0.125 mmol), the Fmoc is deprotected manually with a 20/80 piperidine/NMP mixture and the triBocGlycineHydrazine is coupled, also using HBTU, HOBt and DIEA (4 eq/4 eq/4 eq/8 eq). After controlling the coupling using a Kaiser test, the resin is dried and cleaved for 1 h 30 with 2.75 ml of a phenol/EDT/thioanisole/H₂O/TFA mixture (0.3 g/0.1 ml/0.2 ml/0.2 ml/qs for 4 ml). The peptide is precipitated in 200 ml of a 50/50 Et₂O/pentane mixture. After lyophilization, 42.5 mg of crude peptide are obtained (i.e. a coupling yield of 45.4%).

After purification by RP-HPLC on a C18 nucleosil column (t°=60° C., λ=225 nm, buffer A=H₂O/0.05% TFA, buffer B=40% n-propanol/60% H₂O/0.05% TFA, gradient from 0 to 20% of B in 30 minutes, flow rate of 4 ml/min), freezing and lyophilization, 16.4 mg of pure product are obtained (i.e. a final yield of 17.5%). MALDI-TOF analysis: [M+H]+ calculated 522, observed 522.3.

2) Synthesis of the Oligonucleotide of Formula HCO—CO—HN—C₁₂H₂₄-ATCGATCG

This oligonucleotide is obtained under conventional conditions of periodate oxidation (100 mM phosphate buffer, pH=6.56, reaction for 35 minutes with 1 mM NaIO₄, then reaction stopped with 2 equivalents of tartaric acid relative to NaIO₄), purified under the usual conditions, isolated, frozen and lyophilized, adding mannitol (6.67 μg of mannitol/μg of oligonucleotide) and tri-n-butyl-phosphine (0.005% by volume). The mass of this oligonucleotide was controlled by electrospray mass spectrometry under the usual conditions.

3) Reaction for Ligation between the Oligonucleotide and the Peptide

The reaction for ligation between 6.3 μg of oligonucleotide dissolved in 6 μl of 50 mM citrate buffer, pH=5.33, and 3.5 μg (2 eq) of hydrazino-peptide dissolved in 4 μl of water is initiated. The eppendorf containing the reaction medium, covered with parafilm in order to limit evaporation, is placed in a bath at 37° C. The eppendorf is removed from the thermostated bath and 10 μl of citrate buffer are added 1 hour later. The reaction medium is left at ambient temperature and frozen at −30° C. after 27 h. After thawing, 5 μl of reaction medium are removed and 55 μl of water are added thereto. Analytical RP-HPLC is performed on a 250×4.6 mm C18 hypersil column (t°=30° C., λ=260 nm, buffer A=99/1 100 mM TEAA, pH=6.5/CH₃CN, buffer B=95/5 CH₃CN/H₂O, gradient from 1 to 40% of B in 28 minutes, volume injected=30 μl, flow rate=1 ml/min). A new product effectively appeared. The reaction medium is frozen again while waiting to purify it.

After thawing, the reaction medium is diluted with 300 μl of water and 270 μl are injected onto a 250×4.6 mm C18 hypersil column (t°=30° C., λ=260 nm, buffer A=99/1 100 mM TEAA, pH=6.5/CH₃CN, buffer B=95/5 CH₃CN/mQ H₂O, gradient from 1 to 40% of B in 28 minutes, volume injected=30 μl, flow rate=1 ml/min). The major product is collected. The product is frozen and lyophilized. It is taken up in 250 μl of water and assayed: 0.022 OD/250 μl, i.e. 0.726 μg of oligonucleotide. This product is analyzed by MALDI-TOF. [M+H]+ calculated 3233.6, observed 3233.5. It has the following formula:

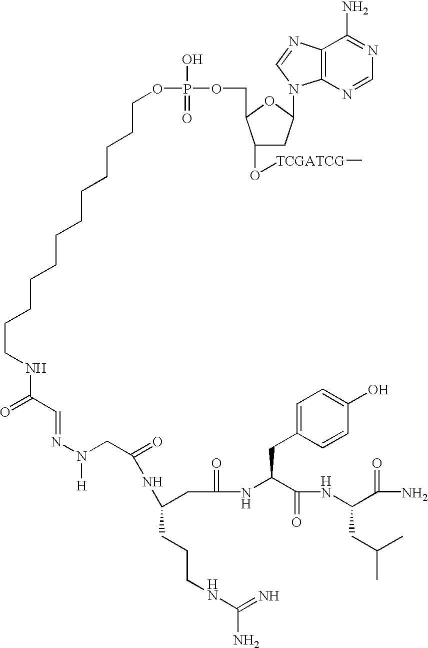

As emerges from the above, the invention is in no way limited to its methods of implementation, preparation and application which have just been described more explicitly; on the contrary, it encompasses all the variants thereof which may occur to a person skilled in the art, without departing from the context or scope of the present invention.

In particular, it is understood that, in formulae (I) and (II) of the products and of the supports according to the present invention, and also in the method for fixing a nucleic acid M to a support SP according to the present invention, the support SP may consist of an arborescent polymer of the polyacrylamide type; in this scenario, it will be advantageous to fix oligonucleotides to this arborescent polymer, by covalent attachment, using the method according to the present invention.

1. A product of formula (I):
SP[Aᵢ(Yᵢ—Z—CO—M)ₙ]ₘ (I)
in which:
Z represents a group of formula 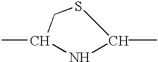
or a group —X—N═CH—, X representing a group —CH₂—O—, —CH₂—NH— or —NH—,
i is equal to 0 or to 1,
n is between 1 and 16, n being equal to 1 when i is equal to 0,
m is greater than or equal to 1,
SP represents a support,
A represents a spacer arm,
Y represents a function which provides the attachment between A and Z, and
M represents a nucleic acid attached to the adjacent group —CO— via its 3′ or 5′ end.

2. The product as claimed in claim 1, characterized in that n is equal to 1 and in that A represents a linear or branched carbon-based chain comprising from 2 to 100 carbon atoms, preferably from 5 to 50 carbon atoms, and optionally comprising from 1 to 35 oxygen or nitrogen atoms and from 1 to 5 silicon, sulfur or phosphorus atoms.

3. The product as claimed in claim 1 or claim 2, characterized in that SP represents a solid support.

4. The product as claimed in claim 3, characterized in that said support is selected from the group consisting of glass, silicon and synthetic polymers.

5. The product as claimed in claim 1 or claim 2, characterized in that said support is a nonsolid support.

6. The product as claimed in claim 5, characterized in that said support is a transfection vector.

7. The product as claimed in any one of claims 1 to 4, characterized in that SP is a solid support, i is equal to 1, n is equal to 1 and M is a DNA, said product constituting a DNA chip.

8. The product as claimed in any one of claims 1 to 4 and 7, characterized in that SP represents a glass support, i is equal to 1, n is equal to 1, A represents a spacer arm of formula —Si—(CH₂)₃— and Y represents an amide function —NH—CO—.

9. The use of the product as claimed in any one of claims 1 to 4, 7 or 8, as a nucleic acid chip.

10. A method for preparing the product of formula (I) as claimed in any one of claims 1 to 8, characterized in that it comprises the reaction of n×m molecules of formula M—CO—CHO with a product of formula SP[Aᵢ(Yᵢ—B—NH₂)ₙ]ₘ, SP, A, Y, i, n, m and M being as defined in any one of claims 1 to 8 and B representing a group —CH₂—O—, —CH₂—NH, —NH— or —CH(CH₂SH)—.

11. A method for fixing, via covalent attachment, at least one nucleic acid M to a support SP, so as to produce a product of formula (I) as claimed in any one of claims 1 to 8, characterized in that it comprises the following steps:
i) introducing an α-oxoaldehyde function onto one end of said nucleic acid, and
ii) reacting the functionalized nucleic acid obtained in step i) with a support modified by a function selected from the group consisting of hydrazine, hydrazine-derived, hydroxylamine and β-aminothiol functions.

12. The method as claimed in claim 11, characterized in that an α-oxoaldehyde function is introduced at one end of said nucleic acid via the following steps:
a) introduction of a group selected from the group consisting of tartaric acid, serine and threonine, and derivatives thereof, at one of the ends of an oligonucleotide,
b) hybridization of the oligonucleotide obtained in step a) with said nucleic acid,
c) elongation of said oligonucleotide,
d) reiteration of steps b) and c) at least once,
e) periodate oxidation of the nucleic acid obtained in step d), modified at one of its ends by a group selected from the group consisting of tartaric acid, serine and threonine, and derivatives thereof, and
f) isolation of a nucleic acid modified at one of its ends by an α-oxoaldehyde function.

13. The method as claimed in claim 11, characterized in that an α-oxoaldehyde function is introduced at one end of said nucleic acid via the following steps:
a) introduction of a group selected from the group consisting of tartaric acid, serine and threonine, and derivatives thereof, at one of the ends of an oligonucleotide,
b) periodate oxidation of the oligonucleotide obtained in step a),
c) hybridization of the oligonucleotide obtained in step b), carrying an α-oxoaldehyde function at one of its ends, with said nucleic acid,
d) elongation of said oligonucleotide,
e) reiteration of steps c) and d) at least once, and
f) isolation of a nucleic acid modified at one of its ends by an α-oxoaldehyde function.

14. The method as claimed in claim 12 or claim 13, characterized in that a group selected from the group consisting of tartaric acid, serine and threonine, and derivatives thereof, is introduced at one of the ends of an oligonucleotide via an amide bond.

15. The method as claimed in claim 14, characterized in that said group selected from the group consisting of tartaric acid, serine and threonine, and derivatives thereof, is attached to the oligonucleotide via a spacer arm attached, via one of its ends, to said oligonucleotide and carrying, at its other end, an amine function.

16. The method as claimed in any one of claims 11 to 15, characterized in that said nucleic acid is a DNA.

17. The method as claimed in claim 16, characterized in that said oligonucleotide defined in claim 12 or in claim 13 is an oligodeoxynucleotide primer.

18. The method as claimed in claim 17, characterized in that said primer is a specific primer.

19. The method as claimed in claim 17, characterized in that said primer is a universal primer.

20. An oligonucleotide modified in the 5′ position by a group selected from the group consisting of tartaric acid, serine and threonine, and derivatives thereof, and the α-oxoaldehyde group.

21. A method for preparing an oligonucleotide as claimed in claim 20, characterized in that it comprises step a) according to claim 13, followed, when said oligonucleotide is modified by an α-oxoaldehyde group, by step b) according to claim 13.

22. A DNA modified in the 5′ position by a group selected from the group consisting of tartaric acid, serine and threonine, and derivatives thereof, and the α-oxoaldehyde group.

23. A method for preparing a DNA as claimed in claim 22, characterized in that it comprises steps a) to d) according to claim 12 or, when said DNA is modified by an α-oxoaldehyde group, steps a) to f) as claimed in claim 12 or claim 13.

24. A functionalized support of formula (II):
SP[Aᵢ(Yᵢ—B—NH₂)ₙ]ₘ (II)
in which SP, A, Y, i, n and m are as defined in any one of claims 1 to 8, and in which B is as defined in claim 10.

25. A method for preparing a functionalized support of formula (II) as claimed in claim 24, in which i is equal to 1, n is equal to 1 and SP represents a glass support, characterized in that it comprises the following steps:
silanizing the glass support,
grafting, onto said silanized glass support, a function selected from the group consisting of the hydrazine, hydrazine-derived, hydroxylamine and β-aminothiol functions.

26. The method as claimed in claim 25, characterized in that said silanizing of the support is carried out using aminopropyltrimethoxysilane.

27. The method as claimed in claim 25 or claim 26, characterized in that said grafting of a hydrazine function is carried out using hydrazinoacetic acid, said grafting of a hydrazine-derived function is carried out using triphosgene and hydrazine, said grafting of a hydroxylamine function is carried out using aminooxyacetic acid and said grafting of a β-aminothiol function is carried out using α-amino-β-mercaptopropionic acid.

28. A method for controlling the quality of the support of formula (II) as claimed in claim 24, characterized in that it comprises the following steps:
bringing the support into contact with a fluorescent probe derivatized with an α-oxoaldehyde function,
washing the support obtained at the end of the previous step, and
analyzing the fluorescence from this support.

29. A method for quantifying the functionality of the support of formula (II) as claimed in claim 24, characterized in that it comprises the following steps:
bringing the support into contact with a fluorescent probe derivatized with an α-oxoaldehyde function,
washing the support obtained at the end of the previous step,
hydrolyzing the attachment between the support and the fluorescent probe, and
measuring the amount of fluorescence released into solution at the end of this hydrolysis.

30. A kit for preparing a DNA chip as claimed in claim 7, characterized in that it comprises the following elements:
at least one functionalized support as claimed in claim 24,
a plurality of oligodeoxynucleotide primers which are modified either in the 3′ position, or in the 5′ position, or in the 3′ position for a part of said primers and in the 5′ position for the other part of said primers, by a group selected from the group consisting of tartaric acid, serine and threonine, and derivatives thereof, and the α-oxoaldehyde group,
reagents and buffers suitable for carrying out reactions of elongation and/or of amplification of said DNA, and
when said oligodeoxynucleotide primers are modified by a group selected from the group consisting of tartaric acid, serine and threonine, and derivatives thereof, reagents suitable for carrying out a periodate oxidation reaction.

31. The use of the DNA chip as claimed in claim 7, in combinatorial chemistry, in particular for high throughput screening of molecules.

32. The use of the DNA chip as claimed in claim 7, as a diagnostic tool.

33. The use of the DNA chip as claimed in claim 7, for sorting molecules.

34. A method for sorting molecules, characterized in that it uses the DNA chip as claimed in claim 7.

35. A sorted molecule, characterized in that it can be obtained using the method as claimed in claim 34.
